(19) United States
(12) Patent Application Publication
(10) Pub. No.: US 2022/0339360 A1
(43) Pub. Date: Oct. 27, 2022

(54) CONTROL MECHANISM FOR PRIMING AN INJECTION DEVICE

(71) Applicant: Coherus Biosciences, Inc., Redwood City, CA (US)

(72) Inventors: Alasdair George Young, San Francisco, CA (US); Gerald Llorach, Belmont, CA (US); Su Ann Teh, Campbell, CA (US); Dmitry Soyfertis, San Francisco, CA (US); Brian Tachibana, San Francisco, CA (US)

(21) Appl. No.: 17/765,294

(22) PCT Filed: Oct. 2, 2020
(86) PCT No.: PCT/US2020/053966

§ 371 Date: Mar. 30, 2022

Related U.S. Application Data
(60) Provisional application No. 62/916,760, filed on Oct. 17, 2019.
(60) Provisional application No. 62/909,998, filed on Oct. 3, 2019.

(51) Int. Cl. A61M 5/31; A61M 5/315

(52) U.S. Cl. CPC A61M 5/3146; A61M 5/31505; A61M 5/3135; A61M 2005/31508

(57) ABSTRACT

An injection device includes a syringe barrel for holding medication, a collar positioned on a proximal end of the syringe barrel, a piston assembly including a piston arranged in the syringe barrel, and a rod member extending through the collar to the piston, and a blocking member arranged on the rod member. The piston assembly is movable distally relative to the syringe barrel in a priming stroke. The piston assembly is movable relative to the collar during the priming stroke of the piston assembly such that the blocking member longitudinally abuts the piston assembly and the collar at an end of the priming stroke of the piston assembly.

### CROSS-REFERENCE TO RELATED APPLICATIONS

This application claims priority to U.S. Provisional Patent Application Ser. No. 62/909,998, filed Oct. 3, 2019, and U.S. Provisional Patent Application Ser. No. 62/916,760, filed Oct. 17, 2019; the entire contents of which are herein incorporated by reference.

### TECHNICAL FIELD

This specification relates to a control mechanism for an injection device.

### BACKGROUND

Injection devices can be manually operated by operators to deliver medications from the injection devices. An injection device can include a piston assembly, and an operator can manually advance the piston assembly by pushing the piston assembly through the injection device. The piston assembly, as it is pushed through the injection device, can apply pressure on medication in the injection device to expel the medication from the injection device. The injection device can be primed before the medication is expelled from the injection device.

### SUMMARY

An injection device with a piston stroke control mechanism can be used for accurately or precisely delivering medication to a subject. An operator can operate a piston assembly of the injection device to prime the injection device, or to initiate delivery of a dose of the medication to the subject. The piston stroke control mechanism of the injection device can provide one or more mechanical stops for controlling movement of a piston assembly of the injection device during the priming operation or during a dose dispensing operation. For example, a mechanical stop can stop movement of the piston assembly as the user operates the piston assembly to prime the injection device or to deliver the dose of the medication to the subject.

Advantages of the foregoing may include, but are not limited to, those described below and herein elsewhere. In some implementations, the injection device can be simple to assemble and inexpensive to produce. For example, the injection device can be made of only a few parts, e.g., eight, seven, six, or fewer parts, and the control mechanism can also be made of only a few parts, e.g., five, four, three, or fewer parts. The few number of parts can simplify assembly of the injection device and can also result in a relatively inexpensive injection device with a control mechanism for controlling movement during priming or during dose delivery. In addition, rather than using a customized syringe, the injection device can use a standard syringe, and the control mechanism can easily interface with the standard syringe. With such a control mechanism, the standard syringe can be easily adapted into an injection device including one or more mechanical stops for priming or dose delivery as described herein.

Furthermore, in some implementations, by being compatible with standard syringes, the control mechanisms described herein can be used with syringes without requiring a redesign of the syringes. For example, the syringes can be standard prefilled syringes that can be acquired off-the-shelf, and a control mechanism can be designed to be compatible with these standard syringes. The syringes need not be specially designed for the control mechanism.

In some implementations, the control mechanisms described herein can reduce the likelihood of operator error. For example, a piston stroke control mechanism as described herein can be used to control an amount of medication delivered in a dose from the injection device. The control mechanism can prevent the operator from delivering too much medication from the injection device and likewise can prevent the operator from delivering too little medication from the injection device. In addition, in some implementations, the control mechanism can prevent the operator from insufficiently priming the injection device by, for example, not sufficiently advancing the piston assembly to expel air from the injection device. Similarly, the control mechanism can also prevent the operator from over-priming the injection device by, for example, advancing the piston assembly too far, thereby expelling medication from the injection device. Rather than relying on visual monitoring of the injection device, the operator can simply advance a piston assembly until a mechanical stop of the control mechanism is engaged.

Furthermore, the injection device can further include features that prevent the operator from accessing a mechanical stop provided by the control mechanism. In this way, the operator cannot prematurely remove the mechanical stop before a particular stroke, e.g., a priming stroke or a dose delivery stroke, is performed completely.

In one aspect, an injection device includes a syringe barrel for holding medication, a collar positioned on a proximal end of the syringe barrel, a piston assembly including a piston arranged in the syringe barrel, and a rod member extending through the collar to the piston, and a blocking member arranged on the rod member. The piston assembly is movable distally relative to the syringe barrel in a priming stroke. The blocking member is movable with the piston assembly during the priming stroke of the piston assembly and configured to abut the collar at an end of the priming stroke of the piston assembly.

In another aspect, an injection device includes a syringe barrel for holding medication, a collar positioned on a proximal end of the syringe barrel, a piston assembly including a piston arranged in the syringe barrel and a rod member extending through the collar to the piston, and a blocking member arranged on the rod member. The piston assembly is movable distally relative to the syringe barrel in a priming stroke. The piston assembly is movable relative to the collar during the priming stroke of the piston assembly such that the blocking member longitudinally abuts the piston assembly and the collar at an end of the priming stroke of the piston assembly.

In another aspect, a priming stroke control mechanism for an injection device is featured. The priming stroke control mechanism includes a collar configured to be attached to the injection device, and a blocking member arranged within the collar and configured to be attached to a piston assembly of the injection device. The blocking member is movable with the piston assembly during the priming stroke of the piston assembly and configured to abut the collar at an end of the priming stroke of the piston assembly.

In another aspect, a priming stroke control mechanism for an injection device includes a collar configured to be attached to the injection device, and a blocking member arranged within the collar and configured to be attached to a piston assembly of the injection device. The blocking member is movable with the piston assembly during a priming stroke of the piston assembly such that the blocking member longitudinally abuts the piston assembly and the collar at an end of the priming stroke of the piston assembly.

In another aspect, a method of operating an injection is featured. The method includes moving, in a priming stroke, a piston assembly distally through a syringe barrel until a blocking member arranged on the piston assembly prevents the piston assembly from moving distally, removing the blocking member from the piston assembly, and moving distally, in a dispense stroke, the piston assembly to dispense medication from the syringe barrel.

In another aspect, a method of administering medication by injection to a subject is featured. The method includes prior to a dispensing stroke, moving, in a priming stroke, a piston assembly distally through a syringe barrel connected to a needle at a distal end of the syringe barrel until a blocking member arranged on the piston assembly prevents the piston assembly from moving distally, removing the blocking member from the piston assembly, contacting the subject with the needle such that the needle pierces a body of the subject, and moving distally, in a dispense stroke, the piston assembly to dispense medication from the syringe barrel into the subject.

In another aspect, a method of operating an injection device is featured. The method includes moving, in a priming stroke, a piston assembly distally through a syringe barrel until a blocking member arranged on the piston assembly prevents the piston assembly from moving distally, moving the blocking member relative to the piston assembly to allow the piston assembly to move distally; and then moving distally, in a dispense stroke, the piston assembly to dispense medication from the syringe barrel.

In another aspect, a method of administering medication by injection to a subject is featured. The method includes prior to a dispensing stroke, moving, in a priming stroke, a piston assembly distally through a syringe barrel connected to a needle at a distal end of the syringe barrel until a blocking member arranged on the piston assembly prevents the piston assembly from moving distally, moving the blocking member from the piston assembly relative to piston assembly to allow the piston assembly to move distally, contacting the subject with the needle such that the needle pierces a body of the subject, and then moving distally, in a dispense stroke, the piston assembly to dispense medication from the syringe barrel into the subject.

Implementations can include one or more of following features, alone or in combination with one another.

In some implementations, the blocking member can be movable with the piston assembly as the piston assembly moves during the priming stroke of the piston assembly.

In some implementations, the blocking member can include a sleeve portion extending around an outer circumference of the rod member of the piston assembly. In some implementations, the blocking member can include a clip attached to the sleeve portion. The clip can be manually operable by a user to remove the sleeve portion from the rod member of the piston assembly. In some implementations, the clip can be configured to be operated after the end of the priming stroke of the piston assembly to allow the piston assembly to be advanced for a dispense stroke. In some implementations, the clip can extend from the sleeve portion in a direction transverse to a longitudinal axis of the injection device.

In some implementations, the blocking member can include a distal facing surface configured to abut the collar at the end of the priming stroke of the piston assembly. In some implementations, the blocking member can include a proximal facing surface configured to abut the piston assembly at the end of the priming stroke. In some implementations, the proximal facing surface can be a first proximal facing surface of the blocking member. The blocking member can further include a second proximal facing surface configured to abut the piston assembly at an end of a dispense stroke of the piston assembly. In some implementations, the blocking member can be slidable along the distal facing surface of the collar from a first position in which the first proximal facing surface is configured to abut the piston assembly to a second position in which the second proximal facing surface is configured to abut the piston assembly. In some implementations, the blocking member can include an opening configured to receive a manually operable portion of the piston assembly at the end of the dispense stroke. The first proximal facing surface of the blocking member can at least partially define the opening of the blocking member. In some implementations, the second proximal facing surface of the blocking member can be distal to the first proximal facing surface of the blocking member. In some implementations, the blocking member can be rotatable relative to the collar to slide from the first position to the second position. In some implementations, the blocking member can be translatable relative to the collar to slide from the first position to the second position. In some implementations, the blocking member can include a clip portion configured to axially lock the blocking member to the collar.

In some implementations, the distal facing surface can be a first distal facing surface of the blocking member, and the blocking member can be movable relative to the collar from a first position to a second position. The first distal facing surface of the blocking member can be configured to abut the proximal facing surface of the collar when the blocking member is in the first position, and a second distal facing surface of the blocking member being configured to abut the proximal facing surface of the collar when the blocking member is in the second position. In some implementations, the blocking member can be movable relative to the piston assembly from the first position to the second position. In some implementations, the blocking member can include a protruding element configured to be received in an opening along the proximal facing surface of the collar as the blocking member is moved from the first position to the second position. In some implementations, the blocking member can include a clip configured to axially lock the blocking member to collar when the blocking member is in the first position, and can be configured to disengage from the collar when the blocking member moves from the first position to the second position. In some implementations, the blocking member can include a protruding element manually operable by a user to move the blocking member relative to the collar. In some implementations, the piston assembly can include a manually operable portion configured to be pressed by a user to advance the piston assembly in a distal direction. The manually operable portion can include an opening through which the protruding element protrudes at the end of the priming stroke. In some implementations, the blocking member can be rotatable and movable axially relative to the collar from the first position to the second position. In some implementations, the blocking member can include a protruding element including the distal facing surface of the blocking member. The protruding element can be slidable along the proximal facing surface of the collar in a direction transverse to a longitudinal axis of the injection device when the blocking member moves from the first position to the second position.

In some implementations, the piston assembly can include a flexible arm extending longitudinally along the rod member. The flexible arm can be configured to be received in an opening of the blocking member. The opening can be configured to prevent the flexible arm from moving radially outwardly away from the rod member. In some implementations, the flexible arm can be configured to move out of the opening of the blocking member as the piston assembly is advanced distally such that the flexible arm abuts the blocking member to prevent proximal movement of the piston assembly.

In some implementations, the piston assembly can include a manually operable flange portion, and the blocking member can be adjacent to the manually operable flange portion.

In some implementations, the blocking member can include a protruding portion protruding from the rod member. The protruding portion can include a surface configured to abut the collar at the end of the priming stroke of the piston assembly. The blocking member can include a pin member extending from the protruding portion through an opening in the rod member of the piston assembly.

In some implementations, the blocking member can include a protruding portion protruding from the rod member. The protruding portion can include a surface configured to abut the collar at the end of the priming stroke of the piston assembly. The blocking member can include a sleeve portion extending from the protruding portion and around an outer circumference of the rod member of the piston assembly.

In some implementations, the collar can include a cover portion arranged on the blocking member such that the blocking member is inaccessible to a user. In some implementations, the collar can include an opening through which the blocking member is accessible. The blocking member can be aligned with the opening at the end of the priming stroke of the piston assembly. In some implementations, the opening can extend laterally through the collar. In some implementations, the blocking member can be removable from the rod member through the opening of the collar.

In some implementations, the piston assembly can be in a primed position at the end of the priming stroke and can be movable, in a dispense stroke, distally relative to the syringe barrel from the primed position to dispense the medication from the syringe barrel.

In some implementations, the blocking member can be configured to abut the collar to prevent the piston assembly from being moved distally in the dispense stroke until the blocking member is removed from the rod member.

In some implementations, a distal facing surface of the piston assembly can be configured to abut the collar at an end of the dispense stroke.

In some implementations, the piston assembly can include a flange portion on a proximal end of the rod member, the flange portion including the distal facing surface.

In some implementations, the piston of the piston assembly can be configured to be spaced apart from a proximal facing surface of an interior portion of the syringe barrel at the end of the dispense stroke.

In some implementations, the collar can include a flange portion attached to the proximal end of the syringe barrel. The flange portion can include a proximal facing surface configured to abut the blocking member at the end of the priming stroke.

In some implementations, the collar can include a cover portion arranged on the blocking member such that the blocking member is inaccessible to a user. The cover portion can include a proximal facing surface configured to abut the piston assembly at an end of a dispense stroke of the piston assembly.

In some implementations, the proximal facing surface of the flange portion can be configured to abut the piston assembly at an end of a dispense stroke of the piston assembly.

In some implementations, the blocking member can include a protruding portion including a surface configured to abut the collar at the end of the priming stroke of the piston assembly, and a pin member extending from the protruding portion through an opening in a rod member of the piston assembly.

In some implementations, the blocking member can include a protruding portion including a surface configured to abut the collar at the end of the priming stroke of the piston assembly, and a sleeve portion extending from the protruding portion and around the piston assembly. In some implementations, before the priming stroke, the blocking member can be inaccessible by a user. Moving, in the priming stroke, the piston assembly distally through the syringe barrel until the blocking member prevents the piston assembly from moving distally can include moving the blocking member such that the blocking member is accessible by a user.

In some implementations, the needle can pierce an eye of the subject.

The details of one or more implementations of the subject matter described in this specification are set forth in the accompanying drawings and the description below. Other potential features, aspects, and advantages will become apparent from the description, the drawings, and the claims.

### BRIEF DESCRIPTION OF THE DRAWINGS

FIG. 1 is a front view of an example of an injection device.

FIGS. 2A to 2E are front views of the injection device of FIG. 1 during use.

FIG. 3 is a front view of a further example of an injection device.

FIGS. 4A to 4E are front views of the injection device of FIG. 3 during use.

FIG. 5 is a front view of a further example of an injection device.

FIGS. 6A to 6E are front views of the injection device of FIG. 5 during use.

FIG. 7 is a front view of a further example of an injection device.

FIGS. 8A to 8E are front views of the injection device of FIG. 7 during use.

FIGS. 9 and 10 are perspective views of examples of piston stroke control mechanisms.

FIG. 11 is a front view of a further example of an injection device including an inset representing a zoomed in portion of the injection device.

FIGS. 12A to 12E are front views of the injection device of FIG. 11 during use.

FIGS. 13A and 13B are perspective and perspective cutaway views, respectively, of a further example of a piston stroke control mechanism.

FIGS. 14A to 14C are top perspective, front perspective cutaway, and rear perspective cutaway views, respectively, of a further example of a piston stroke control mechanism.

FIGS. 15A to 15C are top rear perspective, top front perspective cutaway, and top front perspective cutaway views, respectively, of a further example of a piston stroke control mechanism.

FIGS. 16A to 16C are top rear perspective, top front perspective cutaway, and top front perspective cutaway views, respectively, of a further example of a piston stroke control mechanism.

FIG. 17 is a front perspective transparent view of an injection device including a lockout mechanism.

FIG. 18 is a front view of a piston assembly including a flexible arm.

FIG. 19 is a front view of a piston assembly including multiple flexible arms.

Like reference numbers and designations in the various drawings indicate like elements.

### DETAILED DESCRIPTION

Injection devices can be used to deliver medication to a subject. For example, an injection device can include a piston assembly that can be manually driven by an operator to cause medicament to be expelled from the injection device. As described herein, the injection device can include a piston stroke control mechanism that limits movement of the piston assembly during operation of the injection device. For example, as described herein, the piston stroke control mechanism can limit an amount of movement of the piston assembly during a dispense stroke or a priming stroke of the piston assembly.

As used herein, the term “distal” refers to being directed toward a dispensing end of an injection device. For example, a “distal end” of a component of an injection device would refer to an end of the component toward the dispensing end of the injection device. As used herein, the term “proximal” refers to being directed away from a dispensing end of an injection device. For example, a “proximal end” of a component of an injection device would refer to an end of the component away from the dispensing end of the injection device.

### Example Injection Devices

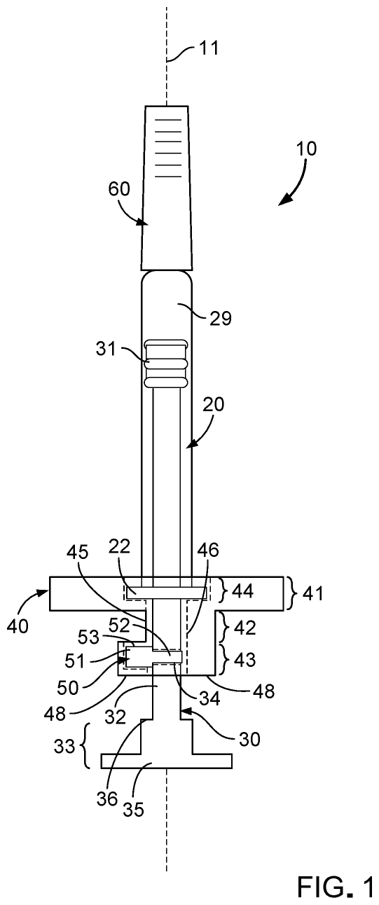

FIG. 1 illustrates an example injection device 10. Referring to FIG. 1, the injection device 10 includes a syringe 20, a piston assembly 30, a collar 40, a blocking member 50, and a needle cover 60. The syringe 20 is arranged with its length along a longitudinal axis 11 of the injection device 10, and the piston assembly 30 is also arranged with its length along the longitudinal axis 11 of the injection device 10. The injection device 10 can be used to administer a single dose of medication to a subject, and then can be disposed of after it is used to administer the single dose. As described herein, the injection device 10 includes a piston stroke control mechanism including features on at least one of the piston assembly 30, the collar 40, or the blocking member 50 to control a dose delivery stroke or a priming stroke of the piston assembly 30. For example, the piston stroke control mechanism can include one or more mechanical stops on the collar 40 to engage with the piston assembly 30 and the blocking member 50 to limit a priming stroke or a dispense stroke of the piston assembly 30. The piston stroke control mechanism can include a priming stroke control mechanism to limit an amount of movement of the piston assembly 30 in the priming stroke, and a dispense stroke control mechanism to limit an amount of movement of the piston assembly 30 in the dispense stroke.

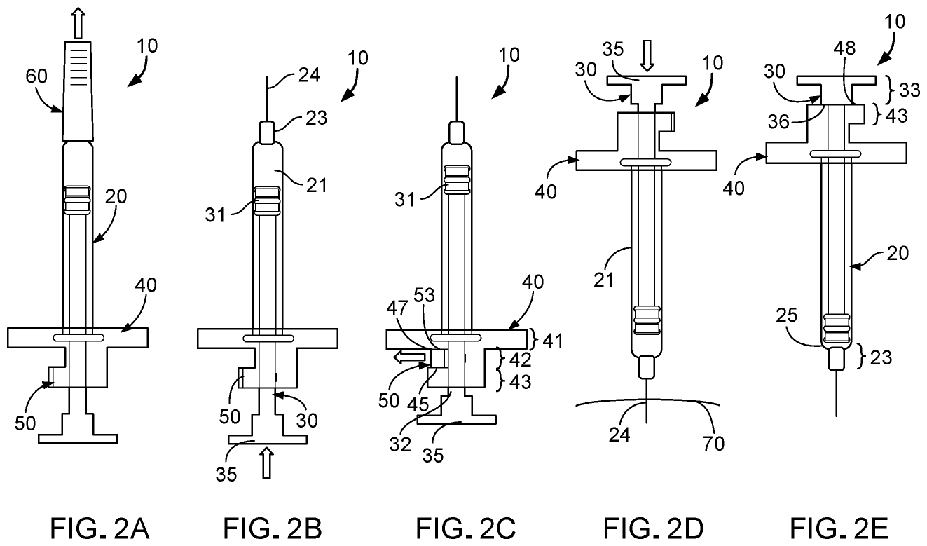

The syringe 20 can include a syringe barrel 21, a flange 22 on a proximal end of the syringe barrel 21, and a narrowed portion 23 (shown in FIG. 2B), e.g., a needle bulb or a needle hub, on a distal end of the syringe barrel 21. The syringe barrel 21 can be a tubular member extending between the flange 22 and the narrowed portion 23. The syringe barrel 21, the flange 22, and the narrowed portion 23 can be unitarily formed of glass, plastic, or another appropriate material for holding medication. In some implementations, a needle 24 (shown in FIG. 2B) is connected to the syringe 20, i.e., connected to the narrowed portion 23 of the injection device 10. In some implementations, the needle 24 is part of a needle assembly separate from the syringe 20. The needle assembly can be removably attached to the syringe 20. The needle 24 provides fluid communication between an environment of the injection device 10 and an interior of the syringe barrel 21 where the medication is stored. The needle 24 extends distally along the longitudinal axis 11 of the injection device 10 from the narrowed portion 23 of the syringe 20. The syringe 20 can be a standard prefilled syringe, e.g., a 0.5 mL, 1.0 mL, 2.0 mL, 2.25 mL syringe.

The piston assembly 30 can include features to form a portion of the piston stroke control mechanism described herein. The piston assembly 30 includes a piston 31 at a distal end of the piston assembly 30, a rod member 32, and a flange portion 33 at a proximal end of the piston assembly 30. The piston assembly 30 is movable distally along the longitudinal axis 11 relative to the syringe barrel 21 in a priming stroke or a dispense stroke.

The piston 31, e.g., a plunger stopper, is slidably arranged within the syringe barrel 21. The piston 31 can be formed of a material that allows the piston 31 to form a seal with the syringe barrel 21. For example, the piston 31 can be formed of an elastomeric material. The syringe barrel 21, the narrowed portion 23, and a piston 31 of the piston assembly can define a space 29 for holding the medication of the injection device 10 before the medication is dispensed from the syringe 20.

The rod member 32 extends proximally from a proximal end of the piston 31 to the flange portion 33. The rod member 32 is an elongate member having a diameter less than a diameter of the piston 31. The rod member 32 transmits force applied to the flange portion 33 to the piston 31. The rod member 32 is attached to the piston 31. For example, the rod member 32 can be threadedly engaged with the piston 31, or engaged with the piston 31 via a press fit. The rod member 32 can include a transverse opening 34 to receive a portion of the blocking member 50. The transverse opening 34 extends in a transverse direction through at least a portion of the rod member 32. In the example shown in FIG. 1, the transverse opening 34 can be a through-hole extending through an entire width of the rod member 32, though in other implementations, the transverse opening 34 may extend only partially through the rod member 32.

The flange portion 33 is manually operable by a human operator of the injection device 10 to dispense medication from the syringe barrel 21. The flange portion 33 is disposed on a proximal end of the rod member 32. The flange portion 33 includes a proximal facing surface 35 and a distal facing surface 36. The proximal facing surface 35 is a manually operable surface of the flange portion 33 that can be pushed by the operator to advance the piston assembly 30 distally relative to the syringe barrel 21. As described herein, the distal facing surface 36 is movable with the piston assembly 30 to abut the collar 40 to limit movement of the piston assembly 30 during a dispense stroke.

The collar 40 can include features to form a portion of the piston stroke control mechanism described herein. The collar 40 can provide one or more mechanical stops to limit movement of the piston assembly 30 in a priming stroke or a dispense stroke. For example, the blocking member 50 can abut the collar 40 to limit movement of the piston assembly 30 in the priming stroke. Furthermore, the piston assembly 30 abuts the collar 40 to limit movement of the piston assembly 30 in the dispense stroke.

The collar 40 is arranged on a proximal end of the syringe 20, e.g., the flange 22 of the syringe 20. The collar 40 can be removably attached to the flange 22. For example, the collar 40 can be an accessory component that can be attached to the syringe 20. The collar 40 includes a distal portion 41, a central portion 42, and a proximal portion 43. A transverse cross-section of the distal portion 41 of the collar 40 (e.g., a cross-section transverse to the longitudinal axis 11 of the injection device 10) can have a shape having a first transverse dimension longer than a second transverse dimension. For example, the transverse cross-section can be rectangular, circular, or elliptical.

The distal portion 41 of the collar 40 can be attached to the flange 22, with the distal portion 41 including a recess 44 into which the flange 22 is inserted. The distal portion 41 is a flange portion of the collar 40 that can be manually handled by the operator, e.g., during a dose delivery operation or a priming operation.

The central portion 42 of the collar 40 extends between the distal portion 41 and the proximal portion 43. The central portion 42 includes a lateral opening 45 extending laterally through the collar 40 and through which, as described herein, the blocking member 50 is accessible to the operator after a priming stroke of the piston assembly 30. For example, referring briefly to FIG. 2C, the blocking member 50 can be movable from a position in the proximal portion 43 to a position in the central portion 42 such that the blocking member 50 is accessible to the operator through the lateral opening 45 of the central portion 42.

Referring back to FIG. 1, the proximal portion 43 is a cover portion arranged over the blocking member 50. The proximal portion 43 of the collar 40 covers the blocking member 50, thereby rendering the blocking member 50 inaccessible to the operator until completion of the priming operation.

The dispense stroke control mechanism of the injection device 10 can include features on the collar 40 and the piston assembly 30. The proximal portion 43 can include a proximal facing surface 48 configured to abut the piston assembly 30, for example, at an end of a dispense stroke. The proximal facing surface 48 on the collar 40 serves as a mechanical stop for the piston assembly at the end of the dispense stroke. For example, the distal facing surface 36 of the flange portion 33 of the piston assembly 30 is movable to a position in which the distal facing surface 36 abuts the proximal facing surface 48 of the proximal portion 43 of the collar 40.

The rod member 32 of the piston assembly 30 extends through the collar 40. For example, the collar 40 can include a longitudinal opening 46 through which the rod member 32 extends. The longitudinal opening 46 can extend along the longitudinal axis 11 of the injection device 10 through the proximal portion 43 and the central portion 42 to the recess 44 in the distal portion 41. In this regard, the longitudinal opening 46 can be a through-opening to accommodate the rod member 32 of the piston assembly 30. The longitudinal opening 46 also can accommodate the blocking member 50, such that the blocking member 50 can be positioned in a proximal portion of the longitudinal opening 46 before a priming stroke, and then can be positioned in a distal portion of the longitudinal opening 46 after the priming stroke. In addition, the longitudinal opening 46 is adjacent to the lateral opening 45, and in particular, the distal portion of the longitudinal opening 46 is continuous with the lateral opening 45.

In some implementations, a width of the distal portion 41 is greater than a width of the flange 22 of the syringe 20. For example, the width of the distal portion 41 is two to four times greater than the width of the flange 22, e.g., two to three times or three to four times greater than the width of the flange 22 of the syringe 20. In this regard, the distal portion 41 of the collar 40, when attached to the syringe 20, can be more easily grasped than the flange 22 of the syringe.

A width of the central portion 42 and a width of the proximal portion 43 of the collar 40 can be less than the width of the distal portion 41. For example, the width of the central portion 42 and the width of the proximal portion 43 can be 25% to 75% of the width of the distal portion 41, e.g., 25% to 50% or 50% to 75% of the width of the distal portion 41.

The blocking member 50 forms a portion of the piston stroke control mechanism described herein. The blocking member 50 includes a protruding portion 51 and a pin member 52 extending laterally from the protruding portion 51. The pin member 52 is inserted into the transverse opening 34 of the rod member 32. With the pin member 52 inserted into the transverse opening 34, the blocking member 50 is coupled to the piston assembly 30 such that the blocking member 50 moves distally with the piston assembly 30 as the piston assembly 30 moves distally.

The protruding portion 51 can be a prismatic or cylindrical structure. The priming stroke control mechanism of the injection device 10 can include features on the blocking member 50 and the collar 40. The protruding portion 51 can be configured to abut the collar 40, for example, at an end of a priming stroke of the piston assembly 30. The protruding portion 51 of the pin member 52 includes a distal facing surface 53 configured to abut the collar 40. For example, the distal facing surface 53 of the protruding portion 51 can abut a proximal facing surface 47 of the distal portion 41 of the collar 40 at, for example, an end of the priming stroke. When the distal facing surface 53 of the protruding portion 51 abuts the proximal facing surface 47 of the distal portion 41, the piston assembly 30 is prevented from moving further distally.

The needle cover 60 is arranged on a distal end of the injection device 10 over the needle 24. The needle cover 60 can engage with the narrowed portion 23 of the syringe 20 to cover the needle 24. The needle cover 60 is removable from the syringe 20, e.g., disengageable from the narrowed portion 23, to allow the needle 24 to be inserted into a subject.

### Example Methods

An example method of using the injection device 10 is presented herein with reference to FIGS. 2A to 2E. Referring to FIG. 2A, the injection device 10 is provided to an operator. In some implementations, the operator can assemble the injection device 10. For example, the collar 40 can be manually attached to the syringe 20 by the operator. In some implementations, the injection device 10 is provided to the operator with the collar 40 already attached to the syringe 20.

The operator then can remove the needle cover 60 to prepare the injection device 10 for a priming operation or a dose delivery operation. The needle cover 60 is, as shown in FIG. 2A, pulled distally away from the syringe 20 to remove the needle cover 60 from the syringe 20. In some implementations, the needle 24 can be attached to the injection device 10. The needle 24 can be part of a needle assembly, and the operator can connect the needle assembly to the injection device.

While in the state shown in FIG. 2A, i.e., before a priming operation is performed, the blocking member 50 is inaccessible to the operator. In particular, the blocking member 50 is arranged under the proximal portion 43 of the collar 40.

After the needle cover 60 is removed and before a dose dispensing operation, referring to FIG. 2B, the operator operates the injection device 10 to perform a priming operation. During the priming operation, the piston assembly 30 is moved in a priming stroke from its initial position (shown in FIG. 2B) to a primed position (shown in FIG. 2C). The operator can perform the priming operation while the injection device 10 is oriented with the needle 24 facing upward such that air within the syringe 20 is positioned near the distal end of the injection device 10. In this way, air in the syringe barrel 21 can be expelled through the needle 24 of the syringe 20 when the piston assembly 30 is advanced distally in the priming stroke. The priming operation can minimize an amount of excess fluid that may be injected into a subject. In some implementations, the injection device 10 is oriented with the needle 24 facing upward during the priming operation.

To perform the priming operation, the operator pushes the proximal facing surface 35 of the piston assembly 30 in a distal direction to move the piston assembly 30 distally through the syringe barrel 21. In addition, the blocking member 50 attached to the piston assembly 30 moves distally with the piston assembly 30 as the piston assembly 30 is advanced distally. As the piston assembly 30 is advanced distally, the piston 31 of the piston assembly 30 applies pressure to fluid within the syringe barrel 21, thereby expelling air from within the syringe barrel 21 through the needle 24.

The operator continues to push the proximal facing surface 35 in the distal direction until, as shown in FIG. 2C, the blocking member 50 attached to the piston assembly 30 abuts the distal portion 41 of the collar 40. For example, the piston assembly 30 can continue to move in the distal direction until the distal facing surface 53 of the blocking member 50 abuts the proximal facing surface 47 of the distal portion 41 of the collar 40. The abutment between the distal facing surface 53 and the proximal facing surface 47 prevents the operator from pushing the piston assembly 30 further in the distal direction.

After the priming operation is performed and the piston assembly 30 is moved in the priming stroke, referring to FIG. 2C, the blocking member 50 is removed from the piston assembly 30. After the priming operation, the blocking member 50 is aligned with the lateral opening 45 of the collar 40, thus allowing the operator to access the blocking member 50 through the collar 40. The operator can pull the blocking member 50 in a transverse direction away from the piston assembly 30. In pulling the blocking member 50 away from the piston assembly 30, the operator removes the pin member 52 (shown in FIG. 1) of the blocking member 50 from the transverse opening 34 (shown in FIG. 1) of the rod member 32 of the piston assembly 30, and pulls the blocking member 50 through the lateral opening 45 of the collar 40.

After the blocking member 50 has been removed from the piston assembly 30, as shown in FIGS. 2D and 2E, the operator performs a dose dispensing operation in which the piston assembly 30 is advanced distally in a dispense stroke. In some implementations, after the priming operation is performed, the needle 24 can be inserted into a subject 70. For example, the operator can move the injection device 10 to a position in which the needle 24 contacts the subject 70, and then can move the injection device 10 in a manner that causes the needle 24 to pierce a body of the subject 70. For example, the injection device 10 can be used to perform an ophthalmic procedure in which medication is delivered to an eye of the subject 70. The needle 24 can thus be maneuvered to pierce a surface of the eye of the subject 70. The needle 24 can be inserted into the subject before the blocking member 50 has been removed from the piston assembly 30 or after the blocking member 50 has been removed.

During the dose dispensing operation, the injection device 10 can be oriented with the needle 24 oriented toward a subject. In FIG. 2D, the piston assembly 30 is in the primed position. The operator can push the proximal facing surface 35 of the piston assembly 30 in the distal direction to advance the piston assembly 30 from the primed position (shown in FIG. 2D) to a dose delivered position (shown in FIG. 2E). As the piston assembly 30 is advanced distally, the piston 31 is also advanced distally through the syringe barrel 21, thereby causing medication in the syringe barrel 21 to be expelled from the syringe barrel 21, out through the needle 24, and into the subject. In particular, the piston 31 of the piston assembly 30 applies pressure to fluid within the syringe barrel 21, thereby expelling medication from within the syringe barrel 21 through the needle 24.

The piston assembly 30 is advanced distally in the dispense stroke until the piston assembly 30 abuts the collar 40. For example, the piston assembly 30 can continue to advance distally until the distal facing surface 36 of the flange portion 33 of the piston assembly 30 abuts the proximal facing surface 48 of the proximal portion 43 of the collar 40. The abutment between the distal facing surface 36 and the proximal facing surface 48 prevents the operator from pushing the piston assembly 30 further in the distal direction.

When the distal facing surface 36 and the proximal facing surface 48 abut one another, the piston assembly 30 is in a dose delivered position. In this regard, when the piston assembly 30 is in the dose delivered position, the piston 31 can be spaced apart from the narrowed portion 23 of the syringe 20. For example, the piston 31 can be spaced apart from a proximal facing surface 25 of an interior of the syringe barrel 21, i.e., a proximal facing surface on the narrowed portion 23. As a result, any force applied by the operator when the piston assembly 30 is in the dose dispensed position is transmitted through the piston assembly 30 to the collar 40 rather than directly to the syringe 20. Such an arrangement can reduce the force exerted on the narrowed portion 23 of the syringe 20, thereby reducing the likelihood of accidental breakage of the syringe 20. In addition, because the piston 31 stops without contacting the narrowed portion 23 of the syringe 20, an amount of movement of the piston 31 during the dispensing operation does not depend on a dimension of the syringe 20, e.g., a length of the syringe 20. The amount of movement of the piston 31 during the dispensing operation depends on parameters of the piston assembly 30 and the collar 40 but does not depend on parameters of the syringe 20. In this regard, the effect of variation on the amount of movement of the piston 31 does not depend on variation in dimensions of the syringe 20. Rather, only variations in dimensions of the piston assembly 30 and the collar 40 can affect this amount of movement. Tolerances for dimensions of the syringe 20 accordingly does not need to be tightly controlled.

After the piston assembly 30 is advanced to the dose dispensed position, the injection device 10 can be discarded.

### Further Alternative Implementations

A number of implementations have been described. Nevertheless, it will be understood that various modifications may be made.

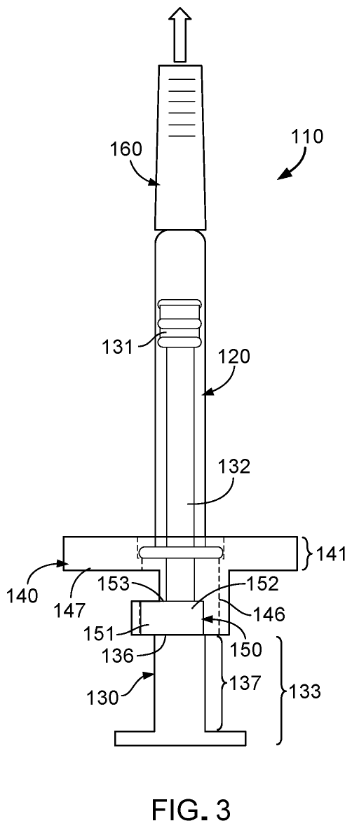

FIG. 3 illustrates another example of an injection device 110 including a syringe 120, a piston assembly 130, a collar 140, a blocking member 150, and a needle cover 160. Similar to the injection device 10, the injection device 110 includes a piston stroke control mechanism including features on at least one of the piston assembly 130, the collar 140, or the blocking member 150 to control a dose delivery stroke or a priming stroke of the piston assembly 130. The piston stroke control mechanism can include a priming stroke control mechanism to limit an amount of movement of the piston assembly 130 for the priming stroke, and a dispense stroke control mechanism to limit an amount of movement of the piston assembly 130 for the dispense stroke. The syringe 120 is similar to the syringe 20 and includes those features described with respect to the syringe 20 shown in FIG. 1.

Like the piston assembly 30 described with respect to FIG. 1, the piston assembly 130 includes a piston 131, a rod member 132, and a flange portion 133. The piston 131 and the rod member 132 are similar to the piston 31 and the rod member 32 and, in some implementations, can include some or all of the features of the piston 31 and the rod member 32.

The flange portion 133 differs from the flange portion 33 in that a distal portion 137 of the flange portion 133 is movable through the collar 140. The distal portion 137 of the flange portion 133 is movable into a longitudinal opening 146 of the collar 140.

The distal portion 137 of the flange portion 133 includes a distal facing surface 136 that abuts with the collar 140 to limit movement of the piston assembly 130 after a dispense stroke. Unlike the distal facing surface 36 of the piston assembly 30 which abuts with the proximal facing surface 47 of the proximal portion 43 of the collar 40, the distal facing surface 136 of the piston assembly 130 abuts a proximal facing surface 147 of a distal portion 141 of the collar 140.

Whereas the blocking member 50 abuts with the distal portion 41 of the collar 40 at an end of the priming stroke, and the piston assembly 30 abuts with the proximal portion 43 of the collar 40 at an end of the dispensing stroke, the collar 140 differs from the collar 40 described with respect to FIG. 1 in that the blocking member 150 and the piston assembly 130 abut the distal portion 141 of the collar 140 in a priming stroke and a delivery stroke, respectively, of the piston assembly 130. At an end of the priming stroke, the proximal facing surface 147 on the distal portion 141 of the collar 140 engages with the distal facing surface 153 of the blocking member 150, and at the end of the delivery stroke, the proximal facing surface 147 engages with a distal facing surface 136 of the piston assembly 130.

In some implementations, as described with respect to FIG. 1, the blocking member 50 includes the pin member 52 to attach the blocking member 50 to the piston assembly 30. In contrast, referring to FIG. 3, the blocking member 150 includes a sleeve portion 152 to attach the blocking member 150 to the piston assembly 130. The sleeve portion 152 is positioned around an outer circumference of the rod member 132 of the piston assembly 130. The sleeve portion 152 can engage with the rod member 132 via a frictional fit, a detent or a groove on the rod member 132 engageable with a front facing surface of the sleeve portion 152, or a hole on the rod member 132 engageable with a pin on the sleeve portion 152. The blocking member 150 also includes a protruding portion 151 protruding from the rod member 132 when the sleeve portion 152 is engaged with the rod member 132.

Like the distal facing surface 53 of the blocking member 50, a distal facing surface 153 of the blocking member 150 can abut the proximal facing surface 147 of the collar 140 at the end of the priming stroke. The distal facing surface 153 of the blocking member 150 can extend across both the protruding portion 151 and the sleeve portion 152. In this regard, both the protruding portion 151 and the sleeve portion 152 of the blocking member 150 can abut the collar 140 at the end of the priming stroke.

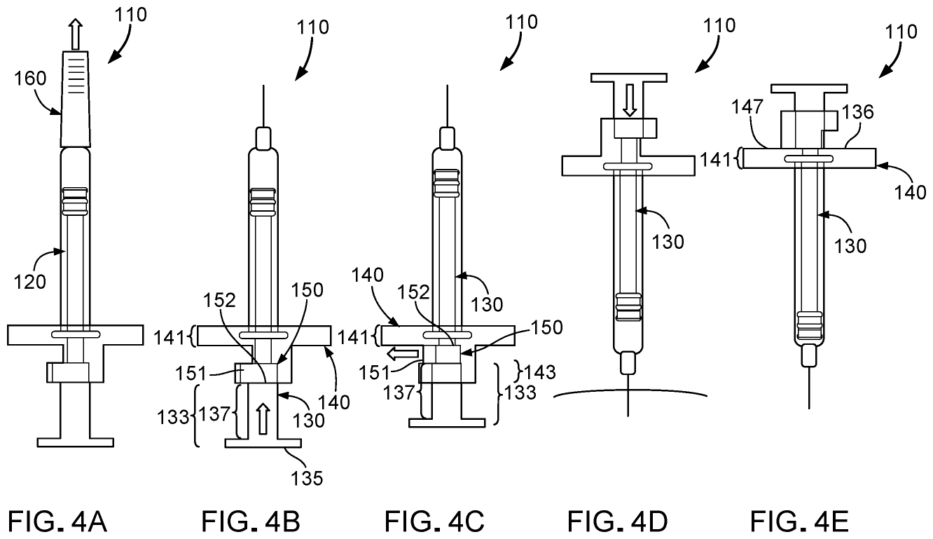

The injection device 110 can be used in a method similar to the method described with respect to the injection device 10 in FIGS. 2A to 2E. FIGS. 4A to 4E illustrate a method for using the injection device 110. First, referring to FIGS. 4A and 4B, the operator removes the needle cover 160 from the syringe 120 like how the needle cover 60 is removed from the syringe 20.

After the needle cover 160 is removed from the syringe 120, the operator performs a priming operation similar to the priming operation performed with the injection device 10. Referring to FIGS. 4B and 4C, to perform the priming operation, the operator pushes a proximal facing surface 135 of the piston assembly 130 to advance the piston assembly 130 in the distal direction in the priming stroke from an initial position (shown in FIG. 4B) to a primed position (shown in FIG. 4C). The priming operation for the injection device 110 differs from the priming operation for the injection device 10 in that the protruding portion 151 and the sleeve portion 152 of the blocking member 150 both abut the distal portion 141 of the collar 140 at the end of the priming stroke. The abutment between the blocking member 150 and the collar 140, in addition to abutment between the piston assembly 130 and the blocking member 150, prevents the operator from pushing the piston assembly 130 further in the distal direction. Furthermore, the distal portion 137 of the flange portion 133 of the piston assembly 130 is movable into the longitudinal opening 146 (shown in FIG. 3) of the collar 140 during the priming operation.

After the priming operation, the operator removes the blocking member 150 from the piston assembly 130. For example, the sleeve portion 152 of the blocking member 150 can include multiple arms that engage with one another to form a sleeve. These arms can be separated from one another to allow the blocking member 150 to be disengaged from the rod member 132.

After the blocking member 150 is removed from the piston assembly 130, the operator performs a dose dispensing operation similar to the dose dispensing operation performed with the injection device 10. Referring to FIGS. 4D and 4E, the operator advances the piston assembly 130 from the primed position (shown in FIG. 4D) to a dose dispensed position (shown in FIG. 4E) in a dispense stroke to dispense medicament from the syringe 120. The dose dispensing operation for the injection device 110 differs from the dose dispensing operation for the injection device 10 in that the distal facing surface 136 of the piston assembly 130 engages with the proximal facing surface of 147 of the distal portion 141 of the collar 140 rather than a proximal portion 143 of the collar 140.

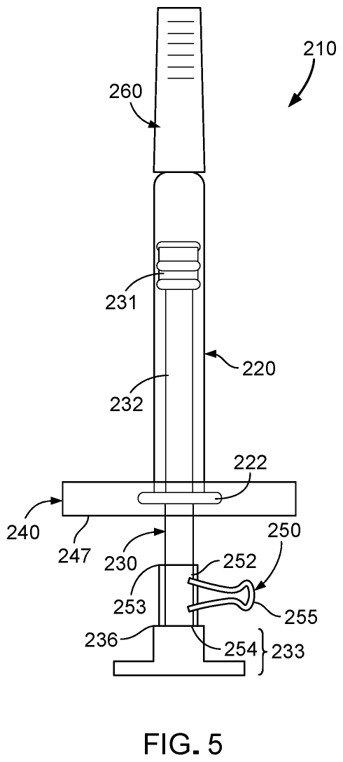

FIG. 5 illustrates another example of an injection device 210 including a syringe 220, a piston assembly 230, a collar 240, a blocking member 250, and a needle cover 260. Similar to the injection device 10, the injection device 210 includes a piston stroke control mechanism including features on at least one of the piston assembly 230, the collar 240, or the blocking member 250 to control a dose delivery stroke or a priming stroke of the piston assembly 230. The piston stroke control mechanism can include a priming stroke control mechanism to limit an amount of movement of the piston assembly 230 for the priming stroke, and a dispense stroke control mechanism to limit an amount of movement of the piston assembly 230 for the dispense stroke. The syringe 220 is similar to the syringe 20 and includes those features described with respect to the syringe 20 shown in FIG. 1.

Like the piston assembly 30 described with respect to FIG. 1, the piston assembly 230 includes a piston 231, a rod member 232, and a flange portion 233. The piston 231 and the rod member 232 are similar to the piston 31 and the rod member 32 and, in some implementations, can include some or all of the features of the piston 31 and the rod member 32. The flange portion 233 can be similar to the flange portion 33 of the piston assembly 30.

The collar 240 is attachable to a flange 222 of the syringe 220. The collar 240 includes a proximal facing surface 247 similar to the proximal facing surface 48 of the collar 40 in that the proximal facing surface 247 can abut a distal facing surface of the blocking member 250 or the piston assembly 230.

In the example shown in FIG. 5, at an end of the priming stroke, the blocking member 250 and the collar 240 longitudinally abut one another, and the blocking member 250 and the piston assembly 230 longitudinally abut one another. For example, the blocking member 250 includes a distal facing surface 253 that abuts the proximal facing surface 247 of the collar 240, and the blocking member 250 includes a proximal facing surface 254 that abuts a distal facing surface 236 of the flange portion 233.

The blocking member 250 is a clip device that can be manually operated by a user to place the blocking member 250 on the piston assembly 230 or remove the blocking member 250 from the piston assembly 230. The blocking member 250, as depicted in FIG. 5, can be arranged on the rod member 232 of the piston assembly 230. The blocking member 250 can include a sleeve portion 252 including the distal facing surface 253 and the proximal facing surface 254. The sleeve portion 252 is positioned around the piston assembly 230. The sleeve portion 252 extends around an outer circumference of the rod member 232 of the piston assembly 230.

In some implementations, the blocking member 250 is loosely positioned on the piston assembly 230. The sleeve portion 252 is positioned around the rod member 232 but is movable along the rod member 232. In this regard, in some implementations, if the injection device 210 is held upside down during the priming stroke, the proximal facing surface 254 of the blocking member 250 abuts the distal facing surface 236 of the flange portion 233 at a start of the priming stroke. At an end of the priming stroke, the distal facing surface 253 abuts the proximal facing surface 247 of the collar 240 to limit the movement of the piston assembly 230. In some implementations, the blocking member 250 is attached to the piston assembly 230 such that the blocking member 250 is movable with the piston assembly 230 as the piston assembly 230 moves during the priming stroke of the piston assembly 230.

The blocking member 250 can include a clip mechanism 255 attached to the sleeve portion 252. The clip mechanism 255 is manually operable by a user to remove the blocking member 250 from the piston assembly 230, e.g., remove the sleeve portion 252 of the blocking member 250 from the rod member 232 from the piston assembly 230. The clip mechanism 255 is configured to be operated after the end of the priming stroke of the piston assembly 230 to allow the piston assembly 230 to be advanced for a dispense stroke. The clip mechanism 255 can include one or more clips that are manually operable by a user to move portions of the sleeve portion 252 away from one another to allow the sleeve portion 252 to be removed from the piston assembly 230. The one or more clips can extend from the sleeve portion 252 in a direction transverse to a longitudinal axis of the injection device 210.

The blocking member 250 can be removed from the piston assembly 230 after the end of the priming stroke. Then, at an end of the dispense stroke, the piston assembly 230 abuts the collar 240 to limit the movement of the piston assembly 230. For example, the distal facing surface 236 of the flange portion 233 can abut the proximal facing surface 247 of the collar 240 to limit the movement of the piston assembly 230 at the end of the dispense stroke.

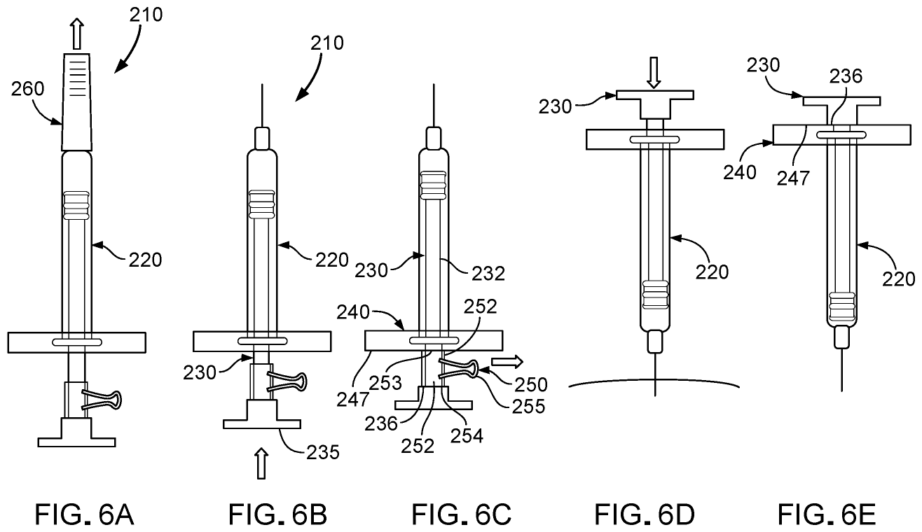

The injection device 210 can be used in a method similar to the method described with respect to the injection device 10 in FIGS. 2A to 2E. FIGS. 6A to 6E illustrate a method for using the injection device 210. First, referring to FIGS. 6A and 6B, the operator removes the needle cover 260 from the syringe 220 like how the needle cover 60 is removed from the syringe 20.

After the needle cover 260 is removed from the syringe 220, the operator uses the injection device 210 to perform a priming operation similar to the priming operation performed with the injection device 10. Referring to FIGS. 6B and 6C, to perform the priming operation, the operator pushes a proximal facing surface 235 of the piston assembly 230 to advance the piston assembly 230 in the distal direction in the priming stroke from an initial position (shown in FIG. 6B) to a primed position (shown in FIG. 6C). As shown in the primed position depicted in FIG. 4C, the priming operation for the injection device 210 results in the distal facing surface 253 of the sleeve portion 252 of the blocking member 250 abutting the proximal facing surface 247 of the collar 240, and the proximal facing surface 254 of the sleeve portion 252 of the blocking member 250 abutting the distal facing surface 236 of the piston assembly 230.

After the priming operation, the operator removes the blocking member 250 from the piston assembly 230. In particular, the operator can operate the clip mechanism 255 of the blocking member 250 to detach the sleeve portion 252 of the blocking member 250 from the rod member 232 of the piston assembly 230.

After the blocking member 250 is removed from the piston assembly 230, the operator performs a dose dispensing operation similar to the dose dispensing operation performed with the injection device 10. Referring to FIGS. 6D and 6E, the operator advances the piston assembly 230 from the primed position (shown in FIG. 6D) to a dose dispensed position (shown in FIG. 6E) in a dispense stroke to dispense medicament from the syringe 220. The distal facing surface 236 of the piston assembly 230 engages with the proximal facing surface 247 of the collar 240 to limit the movement of the piston assembly 230.

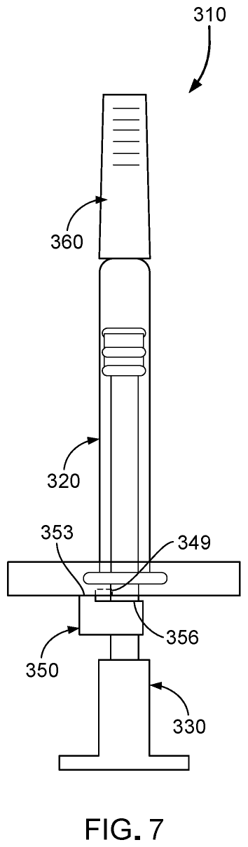

FIG. 7 illustrates another example of an injection device 310 including a syringe 320, a piston assembly 330, a collar 340, a blocking member 350, and a needle cover 360. Similar to the injection device 210, the injection device 310 includes a piston stroke control mechanism including features on at least one of the piston assembly 330, the collar 340, or the blocking member 350 to control a dose delivery stroke or a priming stroke of the piston assembly 230. The piston stroke control mechanism can include a priming stroke control mechanism to limit an amount of movement of the piston assembly 330 for the priming stroke, and a dispense stroke control mechanism to limit an amount of movement of the piston assembly 330 for the dispense stroke. The syringe 320 is similar to the syringe 220 and includes those features described with respect to the syringe 220 shown in FIG. 5.

The injection device 310 differs from the injection device 210 in a few respects. The blocking member 350 is not removed from the injection device 310 between the priming stroke and the dispense stroke. Rather, the blocking member 350 is moved from a first position to a second position between the priming stroke and the dispense stroke.

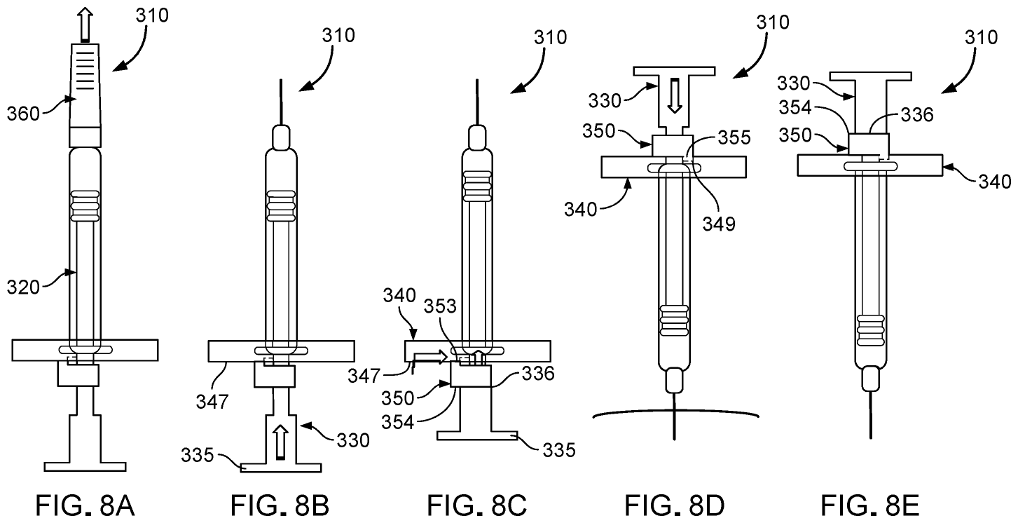

The blocking member 350 is in the first position (shown in FIG. 7) during the priming stroke, and is in the second position (shown in FIG. 8D) during the dispense stroke. At the end of the priming stroke, while the blocking member 350 is in the first position, the piston assembly 330 and the blocking member 350 longitudinally abut one another, and the blocking member 350 and the collar 340 longitudinally abut one another. The blocking member 350 includes at least two distal facing surfaces 353, 356 offset longitudinally from one another. The blocking member 350 includes a protruding element 355 that protrudes distally from the distal facing surface 356 of the blocking member 350. The protruding element 355 abuts longitudinally with the collar 340. The collar 340 includes an opening 349 to receive the protruding element 355 of the blocking member 350. The blocking member 350 is movable, e.g., translatable and/or rotatable, relative to the collar 340 such that the protruding element 355 of the blocking member 350 is received in the opening 349 of the collar 340. In particular, in the first position of the blocking member 350, the distal facing surface 353 of the protruding element 355 abuts the collar 340, and in the second position of the blocking member 350, the distal facing surface 356 of the blocking member 350 abuts the collar 340. At the end of the dispense stroke while the blocking member 350 is in the second position (shown in FIG. 8D), the blocking member 350 and the piston assembly 330 longitudinally abut one another, and the blocking member 350 and the collar 340 longitudinally abut one another. The second position of the blocking member 350 is distal to the first position of the blocking member 350. A longitudinal distance between the first position and the second position can define a length of the dispense stroke.

The injection device 310 can be used in a method similar to the method described with respect to the injection device 210 in FIGS. 6A to 6E. FIGS. 8A to 8E illustrate a method for using the injection device 210. First, referring to FIGS. 8A and 8B, the operator removes the needle cover 360 from the syringe 320 like how the needle cover 260 is removed from the syringe 220.

After the needle cover 360 is removed from the syringe 320, the operator uses the injection device 310 to perform a priming operation similar to the priming operation performed with the injection device 210. Referring to FIGS. 8B and 8C, to perform the priming operation, the operator pushes a proximal facing surface 335 of the piston assembly 330 to advance the piston assembly 330 in the distal direction in the priming stroke from an initial position (shown in FIG. 8B) to a primed position (shown in FIG. 8C). As shown in the primed position depicted in FIG. 8C, at an end of the priming stroke, the distal facing surface 353 of the blocking member 350 abuts a proximal facing surface 347 of the collar 340, and a proximal facing surface 354 of the blocking member 350 abuts a distal facing surface 336 of the piston assembly 330.

In FIGS. 8A-8C, the blocking member 350 is in its first position in which the distal facing surface 353 of the blocking member 350 abuts the proximal facing surface 347 of the collar 340. After the priming operation, as shown in FIG. 8C, the operator moves the blocking member 350 relative to the collar 340 and the piston assembly 330. In particular, the blocking member 350 is moved from its first position (shown in FIG. 8C) to its second position (shown in FIG. 8D). In particular, when the blocking member 350 is moved from the first position to the second position shown in FIG. 8D, the protruding element 355 of the blocking member 350 is moved into the opening 349 of the collar 340. The blocking member 350 is moved in its entirety in the distal direction when moving from the first position to the second position.

After the blocking member 350 is moved to the second position shown in FIG. 8D, referring to FIG. 8E, the operator performs a dose dispensing operation similar to the dose dispensing operation performed with the injection device 210. Referring to FIGS. 8D and 8E, the operator advances the piston assembly 330 from the primed position (shown in FIG. 8D) to a dose dispensed position (shown in FIG. 8E) in a dispense stroke to dispense medicament from the syringe 320. The distal facing surface 336 of the piston assembly 330 abuts the proximal facing surface 354 of the blocking member 350 to limit the movement of the piston assembly 330.

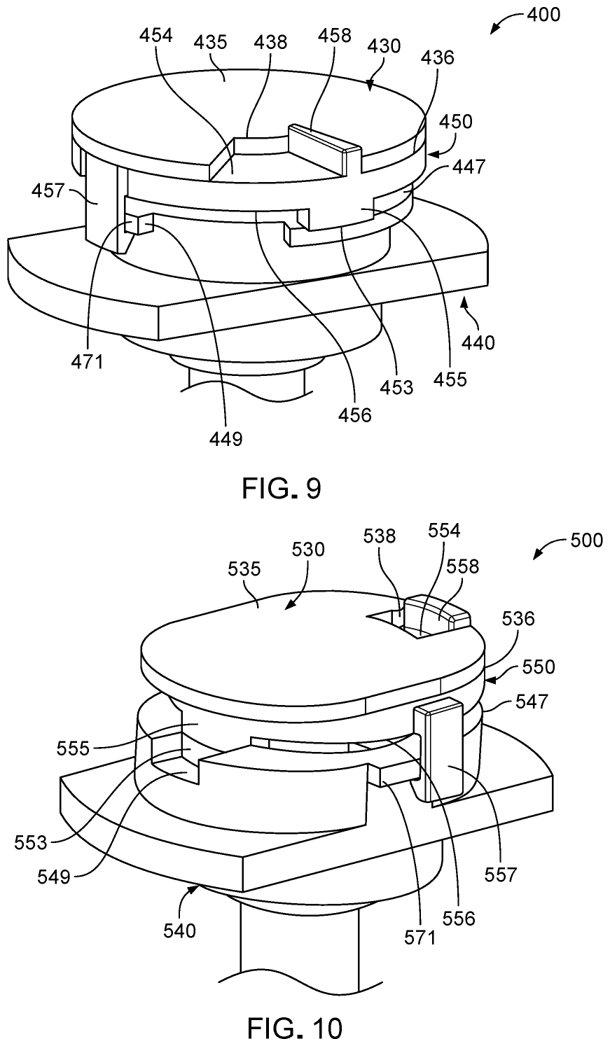

FIG. 9 illustrates an example piston stroke control mechanism 400 similar to the piston stroke control mechanism depicted in FIGS. 7 and 8A to 8E. The piston stroke control mechanism 400 includes a piston assembly 430, a collar 440, and a blocking member 450. The piston assembly 430, the collar 440, and the blocking member 450 are examples of the piston assembly 330, the collar 340, and the blocking member 350 shown in FIGS. 7 and 8A to 8E.

Similar to the collar 340, the collar 440 includes a proximal facing surface 447 (similar to the proximal facing surface 347) and an opening 449 (similar to the opening 349). Similar to the blocking member 350, the blocking member 450 includes a distal facing surface 453 (similar to the distal facing surface 353), a protruding element 455 (similar to the protruding element 355), and a distal facing surface 456 (similar to the distal facing surface 356). In some implementations, the blocking member 450 can include multiple protruding elements similar to the protruding element 455 that are received in corresponding openings of the collar 440 similar to the opening 449. The blocking member 450 further includes a clip portion 457 extending distally away from a remainder of the blocking member 450. The clip portion 457 is engaged with a lip 471 on the collar 440 to prevent the blocking member 450 from moving proximally relative to the collar 440. The clip portion 457 is configured to axially lock the blocking member 450 to the collar 440 when the blocking member 450 is in the first position, and is configured to disengage from the collar 440 when the blocking member moves from the first position to the second position (not shown). In some implementations, the blocking member 450 can include multiple clip portions engaged with corresponding lip portions of the collar 440 to prevent proximal movement of the blocking member 450 relative to the collar 440.

The blocking member 450 can further include a protruding element 458 manually operable by a user to move the blocking member 450 relative to the collar 440 and the piston assembly 430. The protruding element 458 can extend through an opening 438 along a manually operable portion 435 such that the protruding element 458 is manually accessible by a user at the end of the priming stroke when the piston assembly 430 is in the primed position.

FIG. 9 illustrates the blocking member 450 in a first position (similar to the first position of the blocking member 350 shown in FIG. 8C), and illustrates the piston assembly 430 in a primed position (similar to the primed position of the piston assembly 430 shown in FIG. 8C). In the primed position, a distal facing surface 436 of the piston assembly 430 abuts a proximal facing surface 454 of the blocking member 450. The protruding element 458 of the blocking member 450 can be manually operated by the user. Because the protruding element 458 of the blocking member 450 is adjacent to the manually operable portion 435 of the piston assembly 430, the protruding element 458 and the manually operable portion 435 can facilitate single-handed operation of an injection device including the piston stroke control mechanism 400. The protruding element 458 of the blocking member 450 can be pushed from its first position in a manner to rotate the blocking member 450 relative to the collar 440 and relative to the piston assembly 430. The protruding element 455 and its distal facing surface 453 can slide along the proximal facing surface 447 of the collar 440 as the blocking member 450 rotates from the first position toward the second position. The protruding element 455 and the distal facing surface 453 can slide in a direction transverse to a longitudinal axis of the injection device when the blocking member 450 moves from its first position to its second position.

When the protruding element 458 of the blocking member 450 reaches the opening 449 of the collar 440, the blocking member 450 can translate distally relative to the collar 440 and the piston assembly 430 into the second position. The blocking member 450 can translate distally until the distal facing surface 456 abuts the proximal facing surface 447 of the collar 440. When the blocking member 450 is moved to the second position, the proximal facing surface 454 of the blocking member 450 is moved away from the distal facing surface 436 of the piston assembly 430, thus allowing the piston assembly 430 to be advanced distally again for the dispense stroke. When the blocking member 450 is in this second position, the piston assembly 430 can be pushed distally in a dispense stroke to dispense medication from a syringe. A distance between the distal facing surface 456 and the distal facing surface 453 of the blocking member 450 can correspond to a distance of the dispense stroke.

FIG. 10 illustrates another example piston stroke control mechanism 500 similar to the piston stroke control mechanism depicted in FIGS. 7 and 8A to 8E. The piston stroke control mechanism 500 differs from the piston stroke control mechanism 400 in that a blocking member 550 is laterally translatable relative to a collar 540 and a piston assembly 530 to move the blocking member 550 from its first position shown in FIG. 10 toward its second position (not shown).

The blocking member 550 is similar to the blocking member 450 except that the features of the blocking member 550 allow for lateral translation of the blocking member 550 from its first position relative to the collar 540 and the piston assembly 530. The blocking member 550 includes a protruding element 555 including a distal facing surface 553 (similar to the protruding element 455 including the distal facing surface 453), a distal facing surface 556 (similar to the distal facing surface 456), and a protruding element 558 (similar to the protruding element 458). The blocking member 550 further includes a clip portion 557 (similar to the clip portion 457).

The piston assembly 530 is similar to the piston assembly 430. The piston assembly 530 includes a manually operable portion 535 with an opening 538 (similar to the manually operable portion 435 with the opening 438). The collar 540 is similar to the collar 440. The collar 540 includes an opening 549 (similar to the opening 449), a proximal facing surface 547 (similar to the proximal facing surface 447), and a lip 571 (similar to the lip 471).

FIG. 10 illustrates the blocking member 550 in a first position (similar to the first position of the blocking member 450 shown in FIG. 9), and illustrates the piston assembly 530 in a primed position (similar to the primed position of the piston assembly 530 shown in FIG. 9). In the primed position, a distal facing surface 536 of the piston assembly 530 abuts a proximal facing surface 554 of the blocking member 550. The protruding element 558 of the blocking member 550 can be manually operated by the user. Because the protruding element 558 of the blocking member 550 is adjacent to the manually operable portion 535 of the piston assembly 530, the protruding element 558 and the manually operable portion 535 can facilitate single-handed operation of an injection device including the piston stroke control mechanism 500. The protruding element 558 of the blocking member 550 can be pushed from its first position in a manner to translate the blocking member 550 relative to the collar 540 and relative to the piston assembly 530. The protruding element 555 and its distal facing surface 553 can slide along the proximal facing surface 547 of the collar 540 as the blocking member 550 translates from the first position toward the second position. The protruding element 555 and the distal facing surface 553 can slide in a direction transverse to a longitudinal axis of the injection device when the blocking member 550 moves from its first position to its second position.

When the protruding element 558 of the blocking member 550 reaches the opening 549 of the collar 540, the blocking member 550 can translate distally relative to the collar 540 and the piston assembly 530 into the second position. The blocking member 550 can translate distally until the distal facing surface 556 abuts the proximal facing surface 547 of the collar 540. In some implementations, the distal facing surface 553 of the protruding element 555 abuts a surface of the collar 540 in the second position of the blocking member 550. When the blocking member 550 is moved to the second position, the proximal facing surface 554 of the blocking member 550 is moved away from the distal facing surface 536 of the piston assembly 530, thus allowing the piston assembly 530 to be advanced distally again for the dispense stroke. When the blocking member 550 is in this second position, the piston assembly 530 can be pushed distally in a dispense stroke to dispense medication from a syringe. A distance between the distal facing surface 556 and the distal facing surface 553 of the blocking member 550 can correspond to a distance of the dispense stroke.

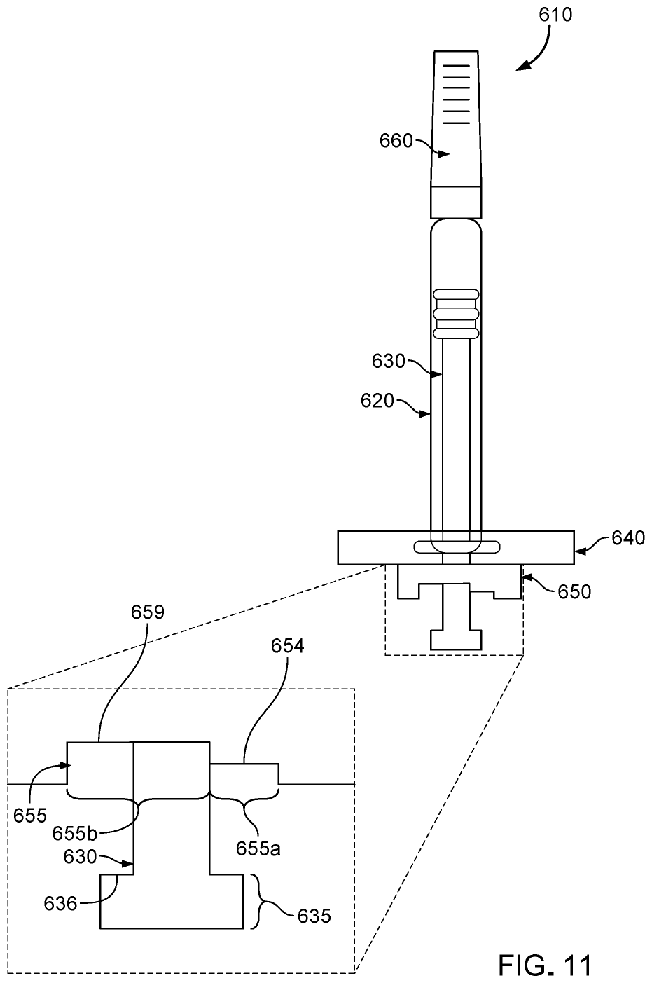

FIG. 11 illustrates another example of an injection device 610 including a syringe 620, a piston assembly 630, a collar 640, a blocking member 650, and a needle cover 660. Similar to the injection device 310, the injection device 610 includes a piston stroke control mechanism including features on at least one of the piston assembly 630, the collar 640, or the blocking member 650 to control a dose delivery stroke or a priming stroke of the piston assembly 330. The piston stroke control mechanism can include a priming stroke control mechanism to limit an amount of movement of the piston assembly 630 for the priming stroke, and a dispense stroke control mechanism to limit an amount of movement of the piston assembly 630 for the dispense stroke. The syringe 620 is similar to the syringe 320 and includes those features described with respect to the syringe 320 shown in FIG. 5.

The injection device 610 differs from the injection device 310 in a few respects. First, the blocking member 650 is not moved distally relative to the piston assembly 630 or the collar 640. Rather, the blocking member 650 is moved from a first position to a second position between the priming stroke and the dispense stroke, and the blocking member 650 is at the same location along a longitudinal axis of the injection device in both the first position and the second position. The blocking member 650 in the second position is laterally offset from the first position but is not vertically offset from the first position.

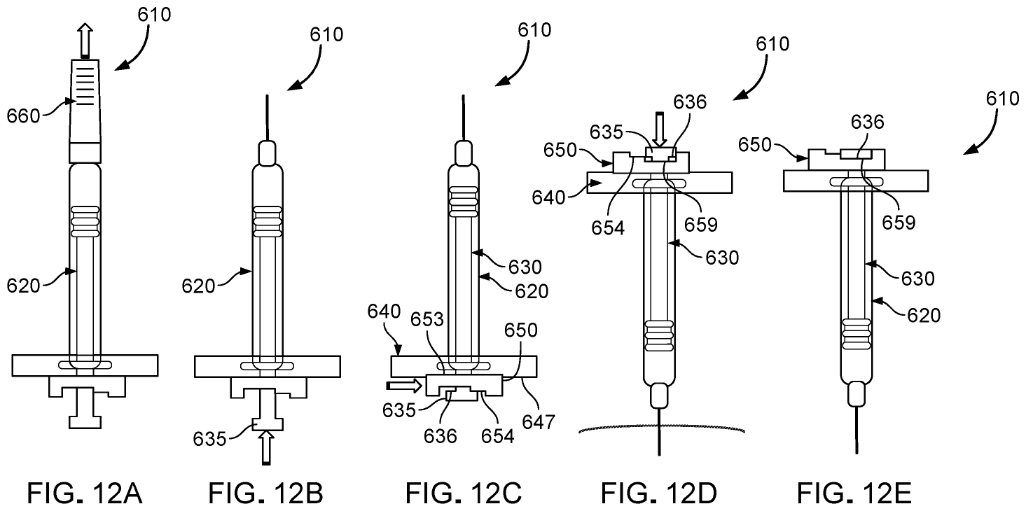

The blocking member 650 is in the first position (shown in FIG. 11) during the priming stroke, and is in the second position (shown in FIG. 12D) during the dispense stroke. At the end of the priming stroke, while the blocking member 650 is in the first position, the piston assembly 630 and the blocking member 650 longitudinally abut one another, and the blocking member 650 and the collar 640 longitudinally abut one another. The blocking member 650 includes at least two proximal facing surfaces 654, 659 offset longitudinally from one another. The proximal facing surface 659 is distal to the proximal facing surface 654. An opening 655 can include the proximal facing surface 654 and the proximal facing surface 659. The opening 655 can include a first portion 655a that is at least partially defined by the proximal facing surface 654 and a second portion 655b that is at least partially defined by the proximal facing surface 659.

The blocking member 650 is movable, e.g., translatable and/or rotatable, relative to the collar 640 such that in the first position of the blocking member 650, the proximal facing surface 654 in the first portion 655a of the opening 655 is configured to abut a distal facing surface 636 of the piston assembly 630 and in the second position of the blocking member 650, the proximal facing surface 659 in the second portion 655b of the opening 655 is configured to abut the distal facing surface 636 of the piston assembly 630. At both the end of the priming stroke while the blocking member 650 is in the first position and the end of the dispense stroke while the blocking member 650 is in the second position, the piston assembly 630 and the blocking member 650 longitudinally abut one another to limit movement of the piston assembly 630. A longitudinal distance between the first position and the second position of the blocking member 650 can define a length of the dispense stroke.

The injection device 610 can be used in a method similar to the method described with respect to the injection device 310 in FIGS. 7 and 8A to 8E. FIGS. 12A to 12E illustrate a method for using the injection device 210. First, referring to FIGS. 12A and 12B, the operator removes the needle cover 660 from the syringe 620 like how the needle cover 360 is removed from the syringe 320.

After the needle cover 660 is removed from the syringe 620, the operator uses the injection device 610 to perform a priming operation similar to the priming operation performed with the injection device 310. Referring to FIGS. 12B and 12C, to perform the priming operation, the operator pushes the manually operable portion 635 of the piston assembly 630 to advance the piston assembly 630 in the distal direction in the priming stroke from an initial position (shown in FIG. 12B) to a primed position (shown in FIG. 12C). As shown in the primed position depicted in FIG. 12C, at an end of the priming stroke, a distal facing surface 653 of the blocking member 650 abuts a proximal facing surface 647 of the collar 640, and the proximal facing surface 654 of the blocking member 650 abuts a distal facing surface 636 of the piston assembly 630. The distal facing surface 653 of the blocking member 650 can abut the proximal facing surface 647 of the collar 640 throughout the priming stroke. At the end of the priming stroke, the manually operable portion 635 of the piston assembly 630 can be received in the opening 655 (shown in FIG. 11).

In FIGS. 12A to 12C, the distal facing surface 636 of the piston assembly 630 abuts the proximal facing surface 654 of the blocking member 650. After the priming operation, as shown in FIG. 12C, the operator moves the blocking member 650 relative to the collar 640 and the piston assembly 630. In particular, the blocking member 650 is moved from its first position (shown in FIG. 12C) to its second position (shown in FIG. 12D). In particular, when the blocking member 650 is moved from the first position to the second position shown in FIG. 12D, the proximal facing surfaces 654 and 659 of the blocking member 650 are moved horizontally relative to the piston assembly 630. The proximal facing surface 654 that was abutting the piston assembly 630 at the end of the piston stroke no longer abuts the piston assembly when the blocking member 650 is in the second position. The proximal facing surface 659 is aligned with the manually operable portion 635 of the piston assembly 630. The blocking member 650 is moved in its entirety in the distal direction when moving from the first position to the second position.

After the blocking member 650 is moved to the second position shown in FIG. 12D, referring to FIG. 12E, the operator performs a dose dispensing operation similar to the dose dispensing operation performed with the injection device 310. Referring to FIGS. 12D and 12E, the operator advances the piston assembly 630 from the primed position (shown in FIG. 12D) to a dose dispensed position (shown in FIG. 12E) in a dispense stroke to dispense medicament from the syringe 620. The distal facing surface 636 of the piston assembly 630 abuts the proximal facing surface 659 of the blocking member 650 to limit the movement of the piston assembly 630 at the end of the dispense stroke.

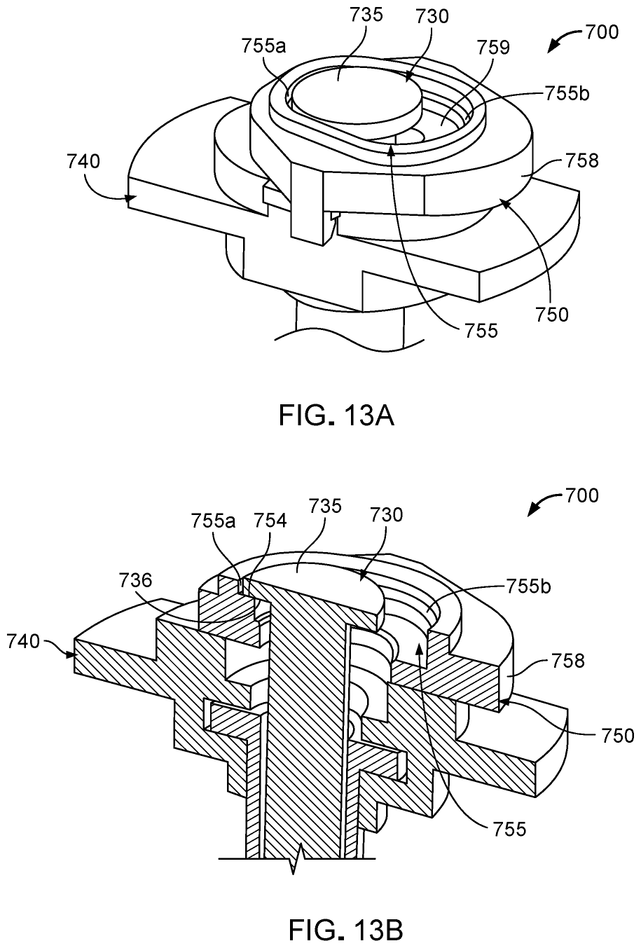

FIGS. 13A-13B illustrate an example piston stroke control mechanism 700 similar to the piston stroke control mechanism depicted in FIGS. 11 and 12A to 12E. The piston stroke control mechanism 700 includes a piston assembly 730, a collar 740, and a blocking member 750. The piston assembly 730, the collar 740, and the blocking member 750 are examples of the piston assembly 630, the collar 640, and the blocking member 650 shown in FIGS. 7 and 8A to 8E.

Similar to the piston assembly 630, the piston assembly 730 includes a manually operable portion 735 (similar to the manually operable portion 635) and a distal facing surface 736 (similar to the distal facing surface 636). Similar to the blocking member 650, the blocking member 750 includes an opening 755 including a first portion 755a and a second portion 755b (similar to the opening 655 including the first portion 655a and the second portion 655b). The blocking member 750 further includes a proximal facing surface 754 (similar to the proximal facing surface 654) and a proximal facing surface 759 (similar to the proximal facing surface 659).

FIGS. 13A-13B show the blocking member 750 in a first position (similar to the first position of the blocking member 650 shown in FIG. 12C), and illustrates the piston assembly 730 in a primed position (similar to the primed position of the piston assembly 730 shown in FIG. 12C). In the primed position, the distal facing surface 736 of the piston assembly 730 abuts the proximal facing surface 754 of the blocking member 750. The manually operable portion 735 of the piston assembly 730 is received in the opening 755 of the blocking member 750. When the manually operable portion 735 is received in the opening 755, a grip portion 758 of the blocking member 750 can be manually operated by the user to slide the blocking member 750 relative to the piston assembly 730 and the collar 740. Because the grip portion 758 of the blocking member 750 is adjacent to the manually operable portion 735 of the piston assembly 730, the grip portion 758 and the manually operable portion 735 can facilitate single-handed operation of an injection device including the piston stroke control mechanism 700. The grip portion 758 of the blocking member 750 can be pushed from its first position in a manner to slide and translate the blocking member 750 relative to the collar 740 and relative to the piston assembly 730. The blocking member 750 can slide along a proximal facing surface of the collar 740 as the blocking member 750 translates from the first position toward the second position. The blocking member 750 can slide in a direction transverse to a longitudinal axis of the injection device when the blocking member 750 moves from its first position to its second position.

After the blocking member 750 is to a second position in which the proximal facing surface 759 is aligned with the manually operable portion 735 of the piston assembly 730, the distal facing surface 736 of the piston assembly 730 no longer abuts the proximal facing surface 754 of the blocking member 750 and thus can be moved distally in a dispense stroke. The distal facing surface 736 can translate distally until the distal facing surface 736 abuts the proximal facing surface 759 of the blocking member 750. The piston assembly 730 can be pushed distally in a dispense stroke to dispense medication from a syringe. A distance between the proximal facing surface 759 and the proximal facing surface 754 of the blocking member 750 can correspond to a distance of the dispense stroke.

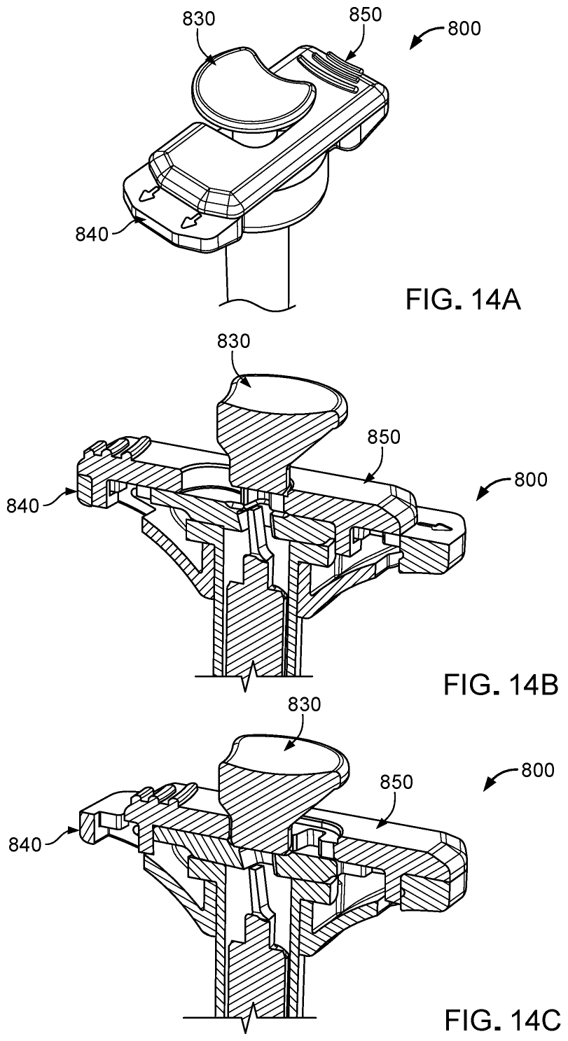

FIGS. 14A-14C illustrate another example piston stroke control mechanism 800 similar to the piston stroke control mechanism depicted in FIGS. 11 and 12A to 12E. The piston stroke control mechanism 800 includes a piston assembly 830, a collar 840, and a blocking member 850. The piston assembly 830, the collar 840, and the blocking member 850 are examples of the piston assembly 730, the collar 740, and the blocking member 750 shown in FIGS. 13A to 13B. FIG. 14B illustrates the piston stroke control mechanism 800 with the blocking member 850 in its first position, and with the piston assembly 830 in the primed position. FIG. 14C illustrates the piston stroke control mechanism 800 with the blocking member 850 in its second position, and with the piston assembly 830 in the dose dispensed position. The blocking member 850, like the blocking member 750, is slidable relative to the collar 840 and the piston assembly 830 to move the blocking member 850 from its first position to its second position. The blocking member 850 is pushed from the first position to its second position.

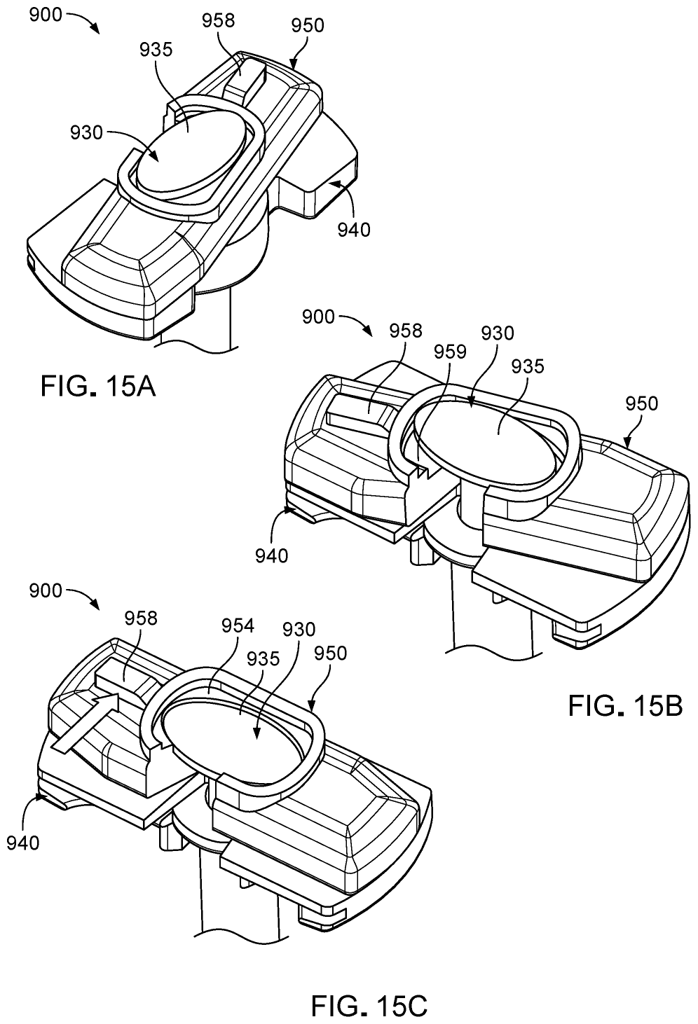

FIGS. 15A to 15C illustrate another example piston stroke control mechanism 900 similar to the piston stroke control mechanism depicted in FIGS. 11 and 12A to 12E. The piston stroke control mechanism 900 includes a piston assembly 930, a collar 940, and a blocking member 950. The piston assembly 930, the collar 940, and the blocking member 950 are examples of the piston assembly 830, the collar 840, and the blocking member 850 shown in FIGS. 14A to 14C. FIG. 15B illustrates the piston stroke control mechanism 900 with the blocking member 950 in its first position, and with the piston assembly 930 in the primed position. FIG. 15C illustrates the piston stroke control mechanism 900 with the blocking member 950 in its second position, and with the piston assembly 930 in the dose dispensed position. The piston stroke control mechanism 900 differs from the piston stroke control mechanism 800 in that the blocking member 950 is rotatable relative to the collar 940 and the piston assembly 930 to move the blocking member 950 from its first position to its second position.

The blocking member 950 can include a protruding element 958 manually operable by a user to rotate the blocking member 950 from its first position to its second position. A manually operable portion 935 of the piston assembly 930 can be a noncircular shape such that in a first position, the manually operable portion 935 of the piston assembly 930 is aligned with a proximal facing surface 954 of the blocking member 950 (similar to the proximal facing surface 754 of the blocking member 750) and in a second position, the manually operable portion 935 of the piston assembly 930 is aligned with a proximal facing surface 959 of the blocking member 950 (similar to the proximal facing surface 759 of the blocking member 750). The manually operable portion 935 can have, for example, an elliptical shape.

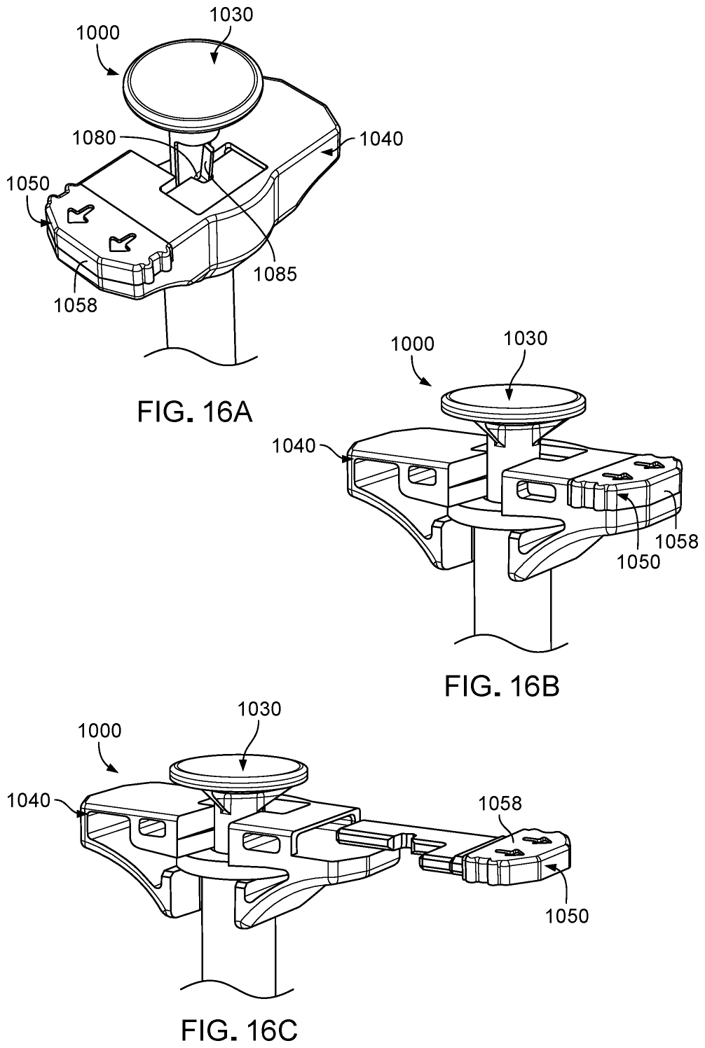

FIGS. 16A to 16C illustrate another example piston stroke control mechanism 1000 similar to the piston stroke control mechanism 800 of FIGS. 14A to 14C. The piston stroke control mechanism 1000 includes a piston assembly 1030, a collar 1040, and a blocking member 1050. The piston assembly 1030, the collar 1040, and the blocking member 1050 are examples of the piston assembly 830, the collar 840, and the blocking member 850 shown in FIGS. 14A to 14C. FIG. 15B illustrates the piston stroke control mechanism 1000 with the blocking member 1050 in its first position, and with the piston assembly 1030 in the primed position. FIG. 15C illustrates the piston stroke control mechanism 1000 with the blocking member 1050 in its second position, and with the piston assembly 1030 in the dose dispensed position. The blocking member 1050, like the blocking member 850, is slidable relative to the collar 1040 and the piston assembly 1030 to move the blocking member 1050 from its first position to its second position. The piston stroke control mechanism 1000 differs from the piston stroke control mechanism 800 in that a portion of the blocking member 1050 is positioned within the collar 1040. The blocking member 1050 and the collar 1040 are an assembly that can be easily attached to a syringe without substantially enlarging a form factor of an injection device. The piston stroke control mechanism 1000 further differs from the piston stroke control mechanism 800 in that a manually operable portion 1058 of the blocking member 1050 is configured to be pulled by the user to be moved from the first position to the second position.

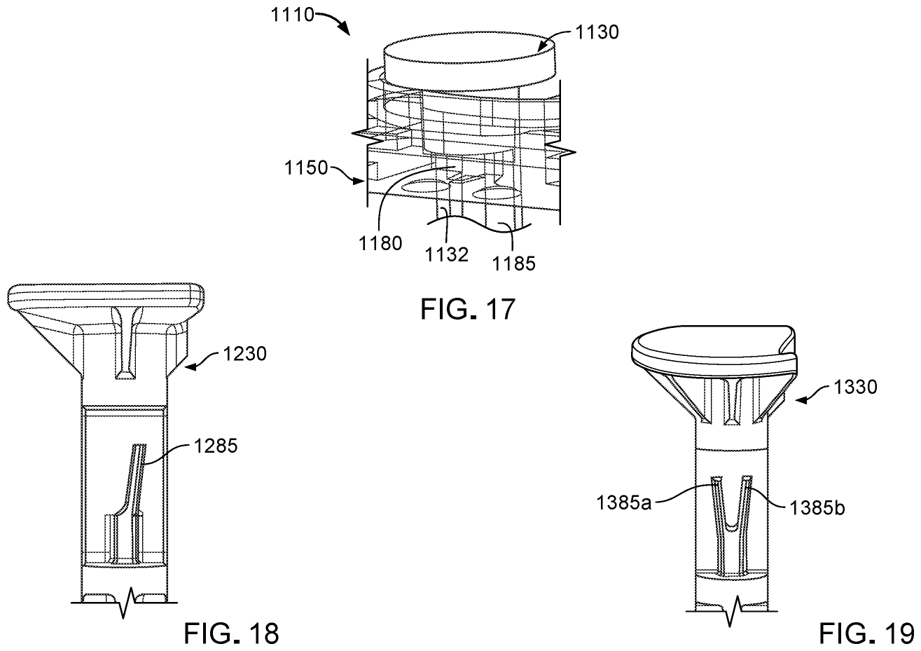

In some implementations, an injection device can include features to prevent the piston assembly from being moved proximally after at least a portion of a priming stroke, a dispense stroke, or both the priming stroke and the dispense stroke. Referring to FIG. 17, an injection device 1110 (similar to any of the examples of the injection devices described herein) includes a lockout mechanism to prevent movement of the piston assembly in a proximal direction. The injection device 1110 includes an opening 1180 in a collar of the injection device 1110 (similar to any of the examples of the collars described herein) or a blocking member 1150 of the injection device 1110 (similar to any of the examples of the blocking members described herein). The opening 1180 can be a feature on the blocking member 1150 as shown in FIG. 17.

A piston assembly 1130 (similar to any of the examples of the piston assemblies described herein) can include a flexible arm 1185 extending longitudinally along a rod member 1132 of the piston assembly 1130. The flexible arm 1185 is receivable in the opening 1180 of the blocking member 1150. The flexible arm 1185 can deflect radially relative to a longitudinal axis of the injection device 1110. In particular, when the flexible arm 1185 is received in the opening 1180, the opening 1180 can prevent the flexible arm 1185 from deflecting radially outwardly away from the rod member 1132. The flexible arm 1185 can be in a deflected state when the flexible arm 1185 is received in the opening 1180. The flexible arm 1185 can be moved back into an undeflected state. In the deflected state, the flexible arm 1185 can abut a portion of the collar or the blocking member 1150. In the example depicted in FIG. 17, a distal end of the flexible arm 1185 abuts a proximal facing surface of the blocking member 1150 to prevent the piston assembly 1130 from being moved proximally relative to the blocking member 1150 and the collar.

Referring back to FIG. 16A, the piston stroke control mechanism 1000 can include a flexible arm 1085 similar to the flexible arm 1185. The flexible arm 1085 extends through an opening 1080 in the blocking member 1050. In the example shown in FIG. 16A, the flexible arm 1085 can be in a deflected state. As the piston assembly 1030 is advanced, the flexible arm 1085 can move to an undeflected state after the flexible arm 1085 moves distally beyond the opening 1080. In the undeflected state, the flexible arm 1085, e.g., an end of the flexible arm 1085, is configured to abut the blocking member 1050 if the piston assembly 1030 is retracted, thus preventing the piston assembly 1030 from being moved proximally relative to the blocking member 1050.

In some implementations, a number of flexible arms on the piston assembly can vary. Referring to FIG. 18, a piston assembly 1230 is similar to the piston assembly 1130 in that the piston assembly 1230 includes a single flexible arm 1285. Referring to FIG. 19, a piston assembly 1330 is similar to the piston assembly 1230 except that the piston assembly 1330 includes multiple flexible arms 1385a, 1385b. Each of the flexible arms 1385a, 1385b can be received in a corresponding opening in the blocking member or the collar of the injection device. In some implementations, a piston assembly can include three or more flexible arms received in corresponding openings in a blocking member or a collar of an injection device.

The injection devices 10, 110 are described as being for ophthalmic procedures. In some implementations, the injection devices 10, 110 can be used for other medical procedures in which medication is delivered to a subject. Non-limiting examples of a medication can include pharmaceutical compositions including one or more of a vascular endothelial growth factor (VEGF) inhibitor (e.g., bevacizumab, ranibizumab, aflibercept, pegaptanib, VEGF Trap-Eye, bevasiranib), triamcinolone acetonide, cortisone, a corticosteroid, dexamethasone, a hyperosmotic solution, erythropoietin, ciprofloxacin, tissue plasminogen activator, an antibiotic (e.g., gentamicin, vancomycin, tobramycin, amikacin, netil-micin, kanamycin, carbenicillin, cefazolin, ofloxacin, lincomycin, azithromycin), rituximab, plasminogen kringle 5, cefazolin, TGF-β, methicillin, microplasmin, a cell (e.g., a mononuclear cell, e.g., a bone marrow-derived mononuclear cell), tissue plasminogen activator, diclonfenac, melphalan, tacrolimus (FK506) (e.g., liposomes encapsulating tacrolimus), ISIS 2922, exendin-4 analogue (E4a), vasoactive intestinal peptide (VIP), cyclosporine (e.g., liposome encapsulated cyclosporine), perfluoropropane (C3F8), amphotericin B, ganciclovir, a cephalosporin, 5-fluorouracil, verteporfin, sulfur hexafluoride, fluconazole, BB-K8, melphalan, cidofovir (HPMPC), methotrexate, hyaluronic acid, rituximab, acyclovir, silicone oil, sodium hyaluronate, voriconazole, tunicamycin, an analgesic (e.g., lidocaine, difluprednate), ceftazidime, infliximab, netarsudil, latanoprost, cenegermin-bkbj, voretigene neparvovec, latanoprostene bunod, cetirizine, adalimumab, lifitegrast, tasimelteon, phenylephrine, ketorolac, cysteamine hydrochloride, ocriplasmin, tafluprost, incobotulinumtoxin A, gatifloxacin, ketorolac tromethamine, bepotastine besilate, besifloxacin, dexamethasone, ganciclovir, bimatoprost, travoprost, valganciclovir, Bexaton, levofloxacin, unoprostone isopropyl, verteporfin, pemirolast potassium, Zaditor, Alrex, Cosopt, Lotemax, Viroptic, Vitravene, ketorolac tromethamine, naphazoline, brimonidine, ofloxacin, OcoHist, cidofovir, and Vitrasert.

The blocking members 50, 150 are described as including a pin member or a sleeve member. The blocking members 50, 150 are removable from the piston assemblies 30, 130 so that the piston assemblies 30, 130 can be advanced for a dose dispensing operation. In some implementations, a blocking member includes other features that allow them to be removed from an injection device. For example, the blocking member can be a breakable component that can be broken so that the blocking member is removable from the piston assembly. The blocking member can include both a pin member and a sleeve member to attach the blocking member to the piston assembly. In some implementations, the blocking member is slidable longitudinally along a rod member of the piston assembly, and the blocking member prevents further movement of the piston assembly when the piston assembly pushes the blocking member against the collar, e.g., a distal facing surface of the piston assembly pushes the blocking member into a proximal facing surface of the collar.

While the injection device 10 is described as being used to administer a single dose of medication, in some implementations, the injection device 10 can be used to administer multiple doses of medication. For example, the piston stroke control mechanism can include multiple mechanical stops on the collar 40, the piston assembly 30, and/or the blocking member 50 to allow for multiple dispense strokes.

The amount of movement in the dispense stroke and the amount of movement in the priming stroke can differ in implementations. In some implementations, the amount of movement in the dispense stroke is between 1 and 6 millimeters, e.g., between 1 and 3 millimeters, 2 and 4 millimeters, 3 and 5 millimeters, 4 and 6 millimeters, or 1.26 and 5.89 millimeters, and the amount of volume of fluid, e.g., medication, liquid, or air, displaced in the dispense stroke is between 40 and 100 microliters, e.g., between 40 and 70 microliters, 50 and 80 microliters, 60 and 90 microliters, or 70 and 100 microliters. The amount of movement in the priming stroke is between 2 and 6 millimeters, e.g., between 2 and 4 millimeters, 3 and 5 millimeters, or 4 and 6 millimeters, and the amount of volume of fluid or medication displaced in the dispense stroke is between 70 and 120 microliters, e.g., between 70 and 100 microliters, 80 and 110 microliters, or 90 and 120 microliters. In some implementations, the amount of movement in the dispense stroke is the same as the amount of movement in the priming stroke. In some implementations, the amount of movement or the amount of volume of fluid displaced in the dispense stroke is less than the amount of movement or the amount of volume of fluid displaced in the priming stroke by, for example, 0.5 to 2 millimeters, e.g., 0.5 to 1.5 millimeters, or 1 to 2 millimeters. In some implementations, the amount of movement or the amount of volume of fluid displaced in the priming stroke is less than the amount of movement or the amount of volume of fluid displaced in the dispense stroke by, for example, 0.5 to 2 millimeters, e.g., 0.5 to 1.5 millimeters, or 1 to 2 millimeters.

While the injection device 10 is described in connection with the syringe 20, in some implementations, the injection device 10 includes a cartridge that can be loaded into a housing of the injection device 10. The cartridge can contain one or more doses of medication to be administered to a subject, and can be arranged with its length along the longitudinal axis 11 of the injection device 10, and is engageable with the piston assembly 30 to dispense the one or more doses from the cartridge. In some implementations, the housing of the injection device 10 can include a distal end engageable with a needle assembly through which the one or more doses can be dispensed. The distal end can be, for example, threadedly engaged with the needle assembly.

The piston assembly 30 is described as including the rod member 32 and the piston 31. In some implementations, the rod member 32 and the piston 31 are detachably engaged with one another. The rod member 32 can be, for example, threadedly engaged with the piston 31.

Also provided herein are methods of using any of the injection devices described herein to inject a therapeutically effective amount of a pharmaceutical composition into the eye of a subject in need thereof.

Also provided herein are kits that include any of the injection devices described herein (e.g., an injection device prefilled with at least one dose of any of the exemplary pharmaceutical compositions described herein or known in the art).

Accordingly, other implementations are within the scope of the claims.

What is claimed is:

1. An injection device comprising:
a syringe barrel for holding medication;
a collar positioned on a proximal end of the syringe barrel;
a piston assembly comprising a piston arranged in the syringe barrel, and a rod member extending through the collar to the piston, wherein the piston assembly is movable distally relative to the syringe barrel in a priming stroke; and
a blocking member arranged on the rod member, wherein the piston assembly is movable relative to the collar during the priming stroke of the piston assembly such that the blocking member longitudinally abuts the piston assembly and the collar at an end of the priming stroke of the piston assembly.

2. The injection device of claim 1, wherein the blocking member is movable with the piston assembly as the piston assembly moves during the priming stroke of the piston assembly.

3. The injection device of claim 1, wherein the piston assembly comprises a manually operable flange portion, and the blocking member is adjacent to the manually operable flange portion.

4. The injection device of claim 1, wherein the blocking member comprises
a protruding portion protruding from the rod member, the protruding portion comprising a surface configured to abut the collar at the end of the priming stroke of the piston assembly, and
a pin member extending from the protruding portion through an opening in the rod member of the piston assembly.

5. The injection device of claim 1, wherein the blocking member comprises
a protruding portion protruding from the rod member, the protruding portion comprising a surface configured to abut the collar at the end of the priming stroke of the piston assembly, and
a sleeve portion extending from the protruding portion and around an outer circumference of the rod member of the piston assembly.

6. The injection device of claim 1, wherein the collar comprises a cover portion arranged on the blocking member such that the blocking member is inaccessible to a user.

7. The injection device of claim 6, wherein the collar comprises an opening through which the blocking member is accessible, wherein the blocking member is aligned with the opening at the end of the priming stroke of the piston assembly.

8. The injection device of claim 7, wherein the opening extends laterally through the collar.

9. The injection device of claim 7, wherein the blocking member is removable from the rod member through the opening of the collar.

10. The injection device of claim 1, wherein the piston assembly is in a primed position at the end of the priming stroke and is movable, in a dispense stroke, distally relative to the syringe barrel from the primed position to dispense the medication from the syringe barrel.

11. The injection device of claim 10, wherein the blocking member is configured to abut the collar to prevent the piston assembly from being moved distally in the dispense stroke until the blocking member is removed from the rod member.

12. The injection device of claim 10, wherein a distal facing surface of the piston assembly is configured to abut the collar at an end of the dispense stroke.

13. The injection device of claim 12, wherein the piston assembly comprises a flange portion on a proximal end of the rod member, the flange portion comprising the distal facing surface.

14. The injection device of claim 12, wherein the piston of the piston assembly is configured to be spaced apart from a proximal facing surface of an interior portion of the syringe barrel at the end of the dispense stroke.

15. The injection device of claim 1, wherein the collar comprises a flange portion attached to the proximal end of the syringe barrel, the flange portion comprising a proximal facing surface configured to abut the blocking member at the end of the priming stroke.

16. The injection device of claim 15, wherein the collar comprises a cover portion arranged on the blocking member such that the blocking member is inaccessible to a user, the cover portion comprising a proximal facing surface configured to abut the piston assembly at an end of a dispense stroke of the piston assembly.

17. The injection device of claim 15, wherein the proximal facing surface of the flange portion is configured to abut the piston assembly at an end of a dispense stroke of the piston assembly.

18. The injection device of claim 1, wherein the blocking member comprises a sleeve portion extending around an outer circumference of the rod member of the piston assembly.

19. The injection device of claim 18, wherein the blocking member comprises a clip attached to the sleeve portion, the clip being manually operable by a user to remove the sleeve portion from the rod member of the piston assembly.

20. The injection device of claim 19, wherein the clip is configured to be operated after the end of the priming stroke of the piston assembly to allow the piston assembly to be advanced for a dispense stroke.

21. The injection device of claim 19, wherein the clip extends from the sleeve portion in a direction transverse to a longitudinal axis of the injection device.

22. The injection device of claim 18, wherein the blocking member comprises a distal facing surface configured to abut the collar at the end of the priming stroke of the piston assembly.

23. The injection device of claim 22, wherein the blocking member comprises a proximal facing surface configured to abut the piston assembly at the end of the priming stroke.

24. The injection device of claim 23, wherein the proximal facing surface is a first proximal facing surface of the blocking member, and the blocking member further comprises a second proximal facing surface configured to abut the piston assembly at an end of a dispense stroke of the piston assembly.

25. The injection device of claim 24, wherein the blocking member is slidable along the distal facing surface of the collar from a first position in which the first proximal facing surface is configured to abut the piston assembly to a second position in which the second proximal facing surface is configured to abut the piston assembly.

26. The injection device of claim 25, wherein the blocking member comprises an opening configured to receive a manually operable portion of the piston assembly at the end of the dispense stroke, wherein the first proximal facing surface of the blocking member at least partially defines the opening of the blocking member.

27. The injection device of claim 26, wherein the second proximal facing surface of the blocking member is distal to the first proximal facing surface of the blocking member.

28. The injection device of claim 25, wherein the blocking member is rotatable relative to the collar to slide from the first position to the second position.

29. The injection device of claim 25, wherein the blocking member is translatable relative to the collar to slide from the first position to the second position.

30. The injection device of claim 23, wherein the blocking member comprises a clip portion configured to axially lock the blocking member to the collar.

31. The injection device of claim 23, wherein:
the distal facing surface is a first distal facing surface of the blocking member,
the blocking member is movable relative to the collar from a first position to a second position, the first distal facing surface of the blocking member being configured to abut the proximal facing surface of the collar when the blocking member is in the first position, and a second distal facing surface of the blocking member being configured to abut the proximal facing surface of the collar when the blocking member is in the second position.

32. The injection device of claim 31, wherein the blocking member is movable relative to the piston assembly from the first position to the second position.

33. The injection device of claim 31, wherein the blocking member comprises a protruding element configured to be received in an opening along the proximal facing surface of the collar as the blocking member is moved from the first position to the second position.

34. The injection device of claim 31, wherein the blocking member comprises a clip configured to axially lock the blocking member to collar when the blocking member is in the first position, and is configured to disengage from the collar when the blocking member moves from the first position to the second position.

35. The injection device of claim 31, wherein the blocking member comprising a protruding element manually operable by a user to move the blocking member relative to the collar.

36. The injection device of claim 35, wherein the piston assembly comprises a manually operable portion configured to be pressed by a user to advance the piston assembly in a distal direction, wherein the manually operable portion comprises an opening through which the protruding element protrudes at the end of the priming stroke.

37. The injection device of claim 31, wherein the blocking member is rotatable and movable axially relative to the collar from the first position to the second position.

38. The injection device of claim 31, wherein the blocking member comprises a protruding element comprising the distal facing surface of the blocking member, the protruding element slidable along the proximal facing surface of the collar in a direction transverse to a longitudinal axis of the injection device when the blocking member moves from the first position to the second position.

39. The injection device of claim 1, wherein the piston assembly comprises a flexible arm extending longitudinally along the rod member, the flexible arm configured to be received in an opening of the blocking member, the opening configured to prevent the flexible arm from moving radially outwardly away from the rod member.

40. The injection device of claim 39, wherein the flexible arm is configured to move out of the opening of the blocking member as the piston assembly is advanced distally such that the flexible arm abuts the blocking member to prevent proximal movement of the piston assembly.

41. A priming stroke control mechanism for an injection device, the priming stroke control mechanism comprising:
a collar configured to be attached to the injection device; and
a blocking member arranged within the collar and configured to be attached to a piston assembly of the injection device, the blocking member movable with the piston assembly during a priming stroke of the piston assembly such that the blocking member longitudinally abuts the piston assembly and the collar at an end of the priming stroke of the piston assembly.

42. The priming stroke control mechanism of claim 41, wherein the blocking member comprises
a protruding portion comprising a surface configured to abut the collar at the end of the priming stroke of the piston assembly, and
a pin member extending from the protruding portion through an opening in a rod member of the piston assembly.

43. The priming stroke control mechanism of claim 41, wherein the blocking member comprises
a protruding portion comprising a surface configured to abut the collar at the end of the priming stroke of the piston assembly, and
a sleeve portion extending from the protruding portion and around the piston assembly.

44. The priming stroke control mechanism of claim 41, wherein the blocking member comprises a proximal facing surface configured to abut the piston assembly at the end of the priming stroke.

45. The injection device of claim 44, wherein the proximal facing surface is a first proximal facing surface of the blocking member, and the blocking member further comprises a second proximal facing surface configured to abut the piston assembly at an end of a dispense stroke of the piston assembly.

46. A method of operating an injection device, the method comprising:
moving, in a priming stroke, a piston assembly distally through a syringe barrel until a blocking member arranged on the piston assembly prevents the piston assembly from moving distally;
moving the blocking member relative to the piston assembly to allow the piston assembly to move distally; and then
moving distally, in a dispense stroke, the piston assembly to dispense medication from the syringe barrel.

47. The method of claim 46, wherein before the priming stroke the blocking member is inaccessible by a user and wherein moving, in the priming stroke, the piston assembly distally through the syringe barrel until the blocking member prevents the piston assembly from moving distally comprises moving the blocking member such that the blocking member is accessible by a user.

48. A method of administering medication by injection to a subject, the method comprising:
prior to a dispensing stroke, moving, in a priming stroke, a piston assembly distally through a syringe barrel connected to a needle at a distal end of the syringe barrel until a blocking member arranged on the piston assembly prevents the piston assembly from moving distally;
moving the blocking member from the piston assembly relative to piston assembly to allow the piston assembly to move distally;
contacting the subject with the needle such that the needle pierces a body of the subject; and then
moving distally, in a dispense stroke, the piston assembly to dispense medication from the syringe barrel into the subject.

49. The method of claim 48, wherein the needle pierces an eye of the subject.
